(19) United States
(12) Patent Application Publication
(10) Pub. No.: US 2009/0169687 A1
(43) Pub. Date: Jul. 2, 2009

(54) Hollow Celery Stick

(75) Inventors: Lawrence K. PIERCE, Aromas, CA (US); Darrell L. DUDA, Palmhurst, TX (US)

Correspondence Address: JONDLE & ASSOCIATES P.C., 858 HAPPY CANYON ROAD SUITE 230, CASTLE ROCK, CO, 80108, US

(73) Assignee: A. Duda & Son, Inc., Oviedo, FL (US)

(21) Appl. No.: 12/398,884

(22) Filed: Mar. 5, 2009

Related U.S. Application Data
(63) Continuation-in-part of application No. 10/423,295, filed on Apr. 25, 2003.

(51) Int. Cl. A23P 1/00; A01H 5/00; A23L 1/212; A23P 1/08

(52) U.S. Cl. 426/106; 800/318; 426/615; 426/518; 426/282

(57) ABSTRACT

An Apium graveolens L. var dulce celery plant with a hollow petiole suitable for use as a straw for consuming beverages or as a food product where other food products are capable of being stuffed inside the hollow celery tube. The edible, hollow celery is mild in taste and more resistant to rupturing than celeriac and leaf celery with hollow petioles.

### CROSS-REFERENCE

This application is a continuation-in-part of U.S. patent application Ser. No. 10/423,295 filed on Apr. 25, 2003 which is herein incorporated by reference in its entirety.

### BACKGROUND OF THE INVENTION

The invention relates to celery products including celery straws and food stuffed celery, and the method that makes these products possible. All publications cited in this application are herein incorporated by reference.

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety or hybrid an improved combination of desirable traits from the parental germplasm. These important traits may include improved flavor, increased stalk size and weight, higher seed yield, improved color, resistance to diseases and insects, tolerance to drought and heat, and better agronomic quality.

Practically speaking, all cultivated forms of celery belong to the species Apium graveolens var. dulce that is grown for its edible stalk. As a crop, celery is grown commercially wherever environmental conditions permit the production of an economically viable yield. In the United States, the principal growing regions are California, Fla., Texas and Michigan. Fresh celery is available in the United States year-round although the greatest supply is from November through January. For planting purposes, the celery season is typically divided into two seasons, summer and winter, with Fla., Texas and the southern California areas harvesting from November to July, and Michigan and northern California harvesting from July to October. Fresh celery is consumed as fresh, raw product and occasionally as a cooked vegetable.

Celery is a cool-season biennial that grows best from 60° F. to 65° F. (16° to 18° C.), but will tolerate temperatures from 45° F. to 75° F. (7° to 24° C.). Freezing will damage mature celery by splitting the petioles or causing the skin to peel, making the stalks unmarketable. This is an occasional problem in plantings in the winter regions. However, celery can tolerate minor freezes early in the crop.

The two main growing regions for celery in California are located along the Pacific Ocean: the central coast or summer production area (Monterey, San Benito, Santa Cruz and San Luis Obispo Counties) and the south coast or winter production area (Ventura and Santa Barbara Counties). A minor region (winter) is located in the southern deserts (Riverside and Imperial Counties).

In the south coast, celery is transplanted from early August to April for harvest from November to mid-July; in the Santa Maria area, celery is transplanted from January to August for harvest from April through December. In the central coast, fields are transplanted from March to September for harvest from late June to late December. In the southern deserts, fields are transplanted in late August for harvest in January.

Commonly used celery varieties for coastal production include Tall Utah 52-75, Conquistador and Sonora. Some shippers use their own proprietary varieties. Celery seed is very small and difficult to germinate. All commercial celery is planted as greenhouse-grown transplants. Celery grown from transplants is more uniform than from seed and takes less time to grow the crop in the field. Transplanted celery is placed in double rows on 100 cm beds with plants spaced between 15.0 cm to 18.0 cm apart.

Celery is an allogamous biennial crop. The celery genome consists of 11 chromosomes. Its high degree of out-crossing is accomplished by insects and wind pollination. Pollinators visiting celery flowers include a large number of wasp, bee and fly species. Celery is subject to inbreeding depression which appears to be genotype dependent, since some lines are able to withstand continuous selfing for three or four generations. Crossing of inbreds results in heterotic hybrids that are vigorous and taller than sib-mated or inbred lines.

Celery flowers are protandrous, with pollen being released 3 to 6 days before stigma receptivity. At the time of stigma receptivity the stamens will have fallen and the two stigmata unfolded in an upright position. The degree of protandry varies, which makes it difficult to perform reliable hybridization, due to the possibility of accidental selfing.

Celery flowers are very small, significantly precluding easy removal of individual anthers. Furthermore, different developmental stages of the flowers in umbels makes it difficult to avoid uncontrolled pollinations. The standard hybridization technique in celery consists of selecting flower buds of the same size and eliminating the older and younger flowers. Then, the umbellets are covered with glycine paper bags for a 5-10 day period, during which the stigmas become receptive. At the time the flowers are receptive, available pollen or umbellets shedding pollen from selected male parents are rubbed on to the stigmas of the female parent.

Celery plants require a period of vernalization while in the vegetative phase in order to induce seed stalk development. A period of 6 to 10 weeks at 5 to 8° C. is usually adequate. However, unless plants are beyond a juvenile state or a minimum of 4 weeks old they may not be receptive to vernalization. Due to a wide range of responses to the cold treatment, it is often difficult to synchronize crossing, since plants will flower at different times. However, pollen can be stored for 6 to 8 months at −10° C. in the presence of silica gel or calcium chloride with a viability decline of only 20 to 40%, thus providing flexibility to perform crosses over a longer time.

For selfing, the plant or selected umbels are caged in cloth bags. These are shaken several times during the day to promote pollen release. Houseflies (Musca domestics) can also be introduced weekly into the bags to perform pollinations.

Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, the overall value of the advanced breeding lines, and the number of successful cultivars produced per unit of input (e.g., per year, per dollar expended, etc.).

Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection.

These processes, which lead to the final step of marketing and distribution, usually take from ten to twenty years from the time the first cross or selection is made. Therefore, development of new cultivars is a time-consuming process that requires precise forward planning, efficient use of resources, and a minimum of changes in direction.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

The goal of plant breeding is to develop new, unique and superior celery cultivars. The breeder initially selects and crosses two or more parental lines, followed by repeated selfing and selection, producing many new genetic combinations. The breeder can theoretically generate billions of different genetic combinations via crossing, selfing and mutations. The breeder has no direct control at the cellular level. Therefore, two breeders will never develop the same line, or even very similar lines, having the same celery traits.

Each year, the plant breeder selects the germplasm to advance to the next generation. This germplasm is grown under unique and different geographical, climatic and soil conditions, and further selections are then made, during and at the end of the growing season. The cultivars that are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce the same line twice by using the exact same original parents and the same selection techniques. This unpredictability results in the expenditure of large research monies to develop superior celery cultivars.

The development of commercial celery cultivars requires the development of celery varieties, the crossing of these varieties, and the evaluation of the crosses. Pedigree breeding and recurrent selection breeding methods are used to develop cultivars from breeding populations. Breeding programs combine desirable traits from two or more varieties or various broad-based sources into breeding pools from which cultivars are developed by selfing and selection of desired phenotypes. The new cultivars are crossed with other varieties and the hybrids from these crosses are evaluated to determine which have commercial potential.

Pedigree breeding is used commonly for the improvement of self-pollinating crops or inbred lines of cross-pollinating crops. Two parents which possess favorable, complementary traits, are crossed to produce an F₁. An F₂ population is produced by selfing one or several F₁'s or by intercrossing two F₁'s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Isozyme Electrophoresis and RFLPs have been widely used to determine genetic composition. Shoemaker and Olsen, (Molecular Linkage Map of Soybean (Glycine max) p 6.131-6.138 in S. J. O'Brien (ed) Genetic Maps: Locus Maps of Complex Genomes, Cold Spring Harbor Laboratory Press, Cold Spring Harbor, N.Y., (1993)) developed a molecular genetic linkage map that consisted of 25 linkage groups with about 365 RFLP, 11 RAPD, three classical markers and four isozyme loci. See also, Shoemaker, R. C., RFLP Map of Soybean, p 299-309, in Phillips, R. L. and Vasil, I. K., eds. DNA-Based Markers in Plants, Kluwer Academic Press, Dordrecht, the Netherlands (1994).

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus in soybean with as many as 26 alleles. (Diwan, N. and Cregan, P. B., Theor. Appl. Genet. 95:22-225, 1997.) SNPs may also be used to identify the unique genetic composition of the invention and progeny varieties retaining that unique genetic composition. Various molecular marker techniques may be used in combination to enhance overall resolution.

Molecular markers, which include markers identified through the use of techniques such as Isozyme Electrophoresis, RFLPs, RAPDs, AP-PCR, DAF, SCARs, AFLPs, SSRs, and SNPs, may be used in plant breeding. One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding is another method of introducing new traits into celery varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

Descriptions of other breeding methods that are commonly used for different traits and crops can be found in one of several reference books (e.g., Principles of Plant Breeding John Wiley and Son, pp. 115-161, 1960; Allard, 1960; Simmonds, 1979; Sneep et al., 1979; Fehr, 1987; “Carrots and Related Vegetable Umbelliferae”, Rubatzky, V. E., et al., 1999).

Proper testing should detect any major faults and establish the level of superiority or improvement over current cultivars. In addition to showing superior performance, there must be a demand for a new cultivar that is compatible with industry standards or which creates a new market. The introduction of a new cultivar will incur additional costs to the seed producer, the grower, processor and consumer for special advertising and marketing, altered seed and commercial production practices, and new product utilization. The testing preceding release of a new cultivar should take into consideration research and development costs as well as technical superiority of the final cultivar. For seed-propagated cultivars, it must be feasible to produce seed easily and economically.

Celery in general is an important and valuable vegetable crop. Thus, a continuing goal of celery plant breeders is to develop stable, high yielding celery cultivars that are agronomically sound. The reasons for this goal are to maximize the amount of yield produced on the land. To accomplish this goal, the celery breeder must select and develop celery plants that have the traits that result in superior cultivars.

The foregoing examples of the related art and limitations related therewith are intended to be illustrative and not exclusive. Other limitations of the related art will become apparent to those of skill in the art upon a reading of the specification.

### SUMMARY OF THE INVENTION

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

It is an aspect of this invention to provide an Apium graveolens L. var dulce celery plant with a hollow celery petiole.

It is another aspect of the present invention to provide an Apium graveolens L. var dulce celery plant with a petiole width between 8.0 mm and 20.0 mm.

It is another aspect of the present invention to provide an Apium graveolens L. var dulce celery plant with a petiole depth between 6.5 mm and 18.5 mm.

It is another aspect of the present invention to provide an Apium graveolens L. var dulce celery petiole with the ability to withstand a vacuum pressure between 12.0 in/hg and 29.0 in/g.

It is another aspect of the present invention to provide an Apium graveolens L. var dulce celery petiole with a wall thickness at the inside petiole cup tissue between 0.67 mm and 2.89 mm.

It is another aspect of the present invention to provide an Apium graveolens L. var dulce celery petiole with a wall thickness at the sidewall of the petiole between 1.50 mm and 5.00 mm.

It is another aspect of the present invention to provide an Apium graveolens L. var dulce celery petiole that has an inside petiole cup tissue with the ability to withstand pressure between 300 grams of pressure and 1300 grams of pressure.

It is still another aspect of the present invention to provide a method for producing an edible celery stick comprising the steps of cutting the celery to remove the leaves, also cutting celery to remove the entire attachment to butt of celery, also removing the heart of celery, cutting celery into limb lengths between 2.0 cm and 36.0 cm, then sanitizing celery and packaging celery.

It is still another aspect of the present invention to provide a method for producing an edible celery stick, wherein cutting the celery is performed by an object selected from the group consisting of knives, razor sharp blades, saws, water jets, lasers and sound waves.

It is still another aspect of the present invention to provide a method for producing an edible celery stick, wherein sanitizing the celery is performed by a sanitization treatment selected from the group consisting of ascorbic acid, peroxyacetic acid, sodium hypochlorite, chlorine, bromine, sodium hypobromine, chlorine dioxide, ozone based systems, hydrogen peroxide products, trisodium phosphate, quaternary ammonium products, ultraviolet light systems, irradiation, steam, ultra heat treatments, and high pressure pasteurization.

It is still another aspect of the present invention to provide a method for producing an edible celery stick, wherein the celery product is packaged in a package selected from the group consisting of flexible film, rigid plastic, solid fiber, poly sleeves, plastic sleeves, poly bags, plastic bags, natural decomposable bags, natural decomposable sleeves, packages that may be opened and resealed, rigid containers like clam shells, packages with different permeability properties, packages with built-in vents and packages with specialized pores or any combination thereof.

It is still another aspect of the present invention to provide a method for injecting consumable material into an edible celery stick, comprising the steps of: providing a food injection device then connecting a food reservoir containing pressurized food to the injection device, then positioning the device into an opening of the hollow celery stick and injecting into the opening of the hollow celery food from the injection device to deliver a quantity of the food.

It is still another aspect of the present invention to provide a method of injecting a consumable material into an edible celery stick wherein said food includes but is not limited to dairy based products, synthetic food types, nut based fillings, soy based products, chocolate, fruits and vegetable products, candy products, ethnic flavorings such as products with Mexican, Japanese, Chinese or Indian flavors or spices, fillings with preservatives, amendments to modify textures such as starches or to control moisture levels, products with nutritional fortification including but not limited to minerals such as calcium and potassium, Vitamins including but not limited to A, C, and D and folic acid.

It is still anther aspect of the present invention wherein the celery stick is battered or coated.

It is still another aspect of the present invention wherein the celery stick is frozen.

It is still another aspect of the present invention wherein the celery stick is grilled, baked or fried.

In addition to the exemplary aspects and embodiments described above, further aspects and embodiments will become apparent by study of the following descriptions.

### DEFINITIONS

In the description and tables which follow, a number of terms are used. In order to provide a clear and consistent understanding of the specification and claims, including the scope to be given such terms, the following definitions are provided:

Allele. Means any of one or more alternative form of a gene, all of which relate to one trait or characteristic. In a diploid cell or organism, the two alleles of a given gene occupy corresponding loci on a pair of homologous chromosomes.

Mean straw width range. Means the range created by the comparison of the mean of the narrowest straw widths as measured in several stalks of hollow celery that can be used as a straw versus the of mean widest widths measured in several stalks of hollow celery sticks that can be used as a straw.

Backcrossing. Means a process in which a breeder repeatedly crosses hybrid progeny back to one of the parents, for example, a first generation hybrid F₁ with one of the parental genotypes of the F₁ hybrid.

Blackheart. Means a lack of movement of sufficient calcium that causes the plant to turn brown and begin to decay at the growing point of the plant. Celery in certain conditions, such as warm weather, grows very rapidly and is incapable of moving sufficient amounts of calcium to the growing point.

Bolting. Means the premature development of a flowering stalk, and subsequent seed, before a plant produces a food crop. Bolting is typically caused by late planting when temperatures are low enough to cause vernalization of the plants.

Bolting Tolerance. Means the amount of vernalization that is required for different celery varieties to bolt is genetically controlled. Varieties with increased tolerance to bolting require greater periods of vernalization in order to initiate bolting. A comparison of bolting tolerance between varieties can be measured by the length of the flowering stem under similar vernalization conditions.

Brown Stem. Means a disease caused by the bacterium Pseudomonas cichorii that causes petiole necrosis. Brown stem is characterized by a firm, brown discoloration throughout the petiole.

Celeriac or Root celery (Apium graveolens L. var. rapaceum). Means a plant that is related to celery but instead of having a thickened and succulent leaf petiole as in celery, celeriac has an enlarged hypocotyl and upper root that is the edible product.

Celery butt. Means the lower portion of the celery plant where the roots and the petioles meet.

Celery Heart. Means the center most interior petioles and leaves of the celery stalk. They are not only the smallest petioles in the stalk, but the youngest as well. Some varieties are considered heartless because they go right from very large petioles to only a couple of very small petioles. The heart is comprised of the petioles that are closest to the meristem of the celery stalk. Most straw and process type varieties have very little heart development.

Consumable. Means material that is considered suitable for eating and palatable by humans.

Crackstem. Means the petiole can crack or split horizontally or longitudinally. Numerous cracks in several locations along the petiole are often an indication that the variety has insufficient boron nutrition. A variety's ability to utilize boron is a physiological characteristic which is genetically controlled.

Dairy products. Means milk-based products that are derived from animal milk including but not limited to cattle, goat or sheep milk.

Dry weight Means the weight of the celery after all water has been removed from celery.

Dry weight percentage. Means the calculation of the dry weight of the celery divided by the original weight of the celery before the removal of the water.

Durable. Means a long-lasting, sturdy and resilient celery, that is able to resist breakage and ruptures through normal harvesting, processing, packaging, shipment and usage.

Edible celery (Apium graveolens L. var. dulce . Means an Apium graveolens L. var. dulce celery that is considered suitable for ingestion by humans based upon the flavor and the texture of the celery.

Efficiency. Means the percentage by weight of the seven-inch sticks compared to the gross weight. More efficient varieties have a greater percentage of the gross weight being converted into useable finished product (i.e., seven-inch sticks).

Essentially all the physiological and morphological characteristics. Means a plant having the physiological and morphological characteristics of the recurrent parent, except for the characteristics derived from the converted gene.

External diameter. Means the average diameter of the petiole cylinder measured from the outside of the cylinder wall to the outside of the opposite cylinder wall.

Feather Leaf. Means a yellowing of the lower leaves. It generally occurs in the outer petioles but can also be found on inner petioles of the stalk. These yellowing leaves which would normally remain in the harvested stalk are considered unacceptable. These petioles then have to be stripped off in order to meet market grade which effectively decreases the stalk size and yield.

Flavor rating. Means a rating based upon mouth flavor or taste.

Furanocoumarins. Means one of the chemical compounds that is responsible for the characteristic flavor and aroma of celery.

Fusarium Yellows. Means a fungal soilborne disease caused by Fusarium oxysporum f. sp. apii Race 2. Infected plants turn yellow and are stunted. Some of the large roots may have a dark brown, water-soaked appearance. The water-conducting tissue (xylem) in the stem, crown, and root show a characteristic orange-brown discoloration. In the later stages of infection, plants remain severely stunted and yellowed and may collapse.

Gross Yield. Means the total yield in pounds per acre, of whole, untrimmed celery plants.

Hollow tube or hollow petiole. Means the shape of the celery petiole wherein the petiole is cylindrical, nearly cylindrical, hemispherical or nearly hemispherical and hollow in the center.

Internal diameter. Means the average diameter of the petiole cylinder measured from the inside of the cylinder wall to the inside of the opposite cylinder wall.

Leaf Celery (Apium graveolens L. var. secalinum). Means a plant that has been developed primarily for leaf and seed production. Often grown in Mediterranean climates, leaf celery more closely resembles celery's wild ancestors. The stems are small and fragile and vary from solid to hollow and the leaves are fairly small and are generally bitter. This type is often used for its medicinal properties and spice.

Limb or celery stick. As used herein “limb” or “celery stick” is the petiole excluding the leaves and the attachment to the butt. It may or may not include the flare or the joint. A celery branch is another term used to describe a celery limb. The celery limb ranges from 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, to 10.0, 10.1, 10.2, 10.3, 10.4, 10.5, 10.6, 10.7, 10.8, 10.9, 11.0, 11.1, 11.2, 11.5, 11.8, 12.3, 12.5, 12.7, 12.9, 13.4, 13.8, 14.2, 14.7, 15.1, 15.8, 16.2, 16.6, 17.3, 17.6, 17.9, 18.5, 18.6, 18.9, 19.2, 19.4, 19.7, 20.0, 20.3, 21.0, 21.5, 21.7, 21.9, 22.3, 22.5, 22.8, 23.6, 23.9, 24, 1, 24.8, 25.6, 25.9, 26.8, 26.9, 27.4, 27.8, 28.5, 28.7, 29.0, 29.2, 29.9, 30.4, 31.5, 32.3, 32.8, 33.4, 33.8, 33.9, 34.6, 35.4 to 36.0 centimeters or higher and including all integers and fractions thereof.

Leaf Margin Chlorosis. Means a magnesium deficiency producing an interveinal chlorosis which starts at the margin of leaves.

Maturity Date. Maturity in celery can be dictated by two conditions. The first, or true maturity, is the point in time when the celery reaches maximum size distribution, but before defects such as pith, yellowing, Feather Leaf or Brown Stem appear. The second, or market maturity is an artificial maturity dictated by market or product conditions, i.e, the market or product requirement may be for smaller diameter straws so the field is harvested at slightly below maximum yield potential because the smaller, younger celery produces smaller straw sizes which the customers may prefer at that moment.

MUN. MUN refers to the MUNSELL Color Chart which publishes an official color chart for plant tissues according to a defined numbering system. The chart may be purchased from the Macbeth Division of Kollmorgen Instruments Corporation, 617 Little Britain Road, New Windsor, N.Y. 12553-6148.

Packaged. Means the celery limbs are packaged according to length and may be packaged in any number of methods according to the specifications of the customer. The product may be packaged and sealed in flexible films, including sleeves or bags that may or may not be resealable, rigid plastic containers like clam shells, solid fiber containers, poly sleeves, plastic sleeves, poly bags, plastic bags, natural decomposable bags, natural decomposable sleeves, or any combination thereof. Variations in the packaging may include different gas exchange rates which may occur due to different permeability or transmission properties of the package materials themselves or due to vents or specialized pores built into the packaging. The package weight ranges from 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 087, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, to 1.60 pounds or higher and including all integers and fractions thereof.

Petiole. Means the stem or limb of a leaf, the primary portion of the celery consumed.

Petiole depth. Means the average measurement in millimeters of the depth of the celery petiole at its narrowest point. The petiole depth measurement is taken from the outside of the petiole (which is the part of the petiole that faces the outside of the stalk) and is measured to the inside of the petiole or cup or the inner most point of the petiole that faces the center of the stalk or heart.

Inside Petiole cup tissue or petiole tissue enclosure. Means the tissue on the inside cup of the petiole that encloses the edges of the petiole cup and creates the hollow celery.

Petiole width. Means the average measurement of the width of the celery petiole in millimeters at its widest point. The measurement is taken from the side or edge of petiole to the opposite side or edge of the petiole. The measurement is taken 90 degrees from petiole depth.

Phthalides. Means one of the chemical compounds that is responsible for the characteristic flavor and aroma of celery.

Plant Height. Means the height of the plant from the bottom of the base or butt of the celery plant to the top of the tallest leaf.

Quantitative Trait Loci. Refers to genetic loci that control to some degree, numerically representable traits that are usually continuously distributed.

Regeneration. Means the development of a plant from tissue culture.

Ribbing. The texture of the surface of the celery petiole can vary from smooth to ribby depending on the variety. Ribbing is the presence of numerous ridges that run vertically along the petioles of the celery plant.

Sanitized. Means washed, cleansed or sterilized hollow celery so the limb's surface is free of dirt, insects, microbial infestation, bacterial infestation, fungal infestation or other surface contaminates. The process of sanitization involves washing the limbs in order to remove surface contamination such as dirt and insects and the utilization of a sanitization material or process in order to remove or kill surface contamination by microbial, bacterial and fungal agents.

Sanitization Treatment. Means treating the celery with a chemical or process so as to sanitize the celery. The chemical or process is selected from the group consisting of ascorbic acid, peroxyacetic acid also known as TSUNAMI, sodium hypochlorite (chlorine), bromine products (sodium hypobromine), chlorine dioxide, ozone based systems, hydrogen peroxide products, trisodium phosphate, quaternary ammonium products, ultraviolet light systems, irradiation, steam, ultra heat treatments, and high pressure pasteurization.

Shear strength or pressure. Means the force in grams that a celery can withstand prior rupturing or cutting of the wall of the celery petiole.

Single gene converted. Means plants which are developed by a plant breeding technique called backcrossing or via genetic engineering wherein essentially all of the desired morphological and physiological characteristics of a line are recovered in addition to the single gene transferred into the line via the backcrossing technique or via genetic engineering.

Soybean product. Means products that are derived from soybeans including but not limited soycheese, soymilk and soynut butter.

Stalk. Means a single celery plant that is trimmed with the top or foliage and having the roots removed.

Straw celery. Means an Apium graveolens L. var. dulce hollow celery stick that is capable of being used to suck up a liquid.

Stringiness. Means a physiological characteristic that is generally associated with strings that get stuck between the consumer's teeth. There are generally two sources of strings in celery. One is the vascular bundle which can be fairly elastic and behave as a string. The second is a strip of particularly strong epidermis which is located on the surface of the ridges of the celery varieties that have ribs.

Stuffed celery. Means an Apium graveolens L. var. dulce hollow celery stick that is capable of being filled with edible products.

Suckers. Means auxiliary shoots that form at the base of the stalk or within the auxiliary buds between each petiole. If these shoots form between the petioles of the stalk, several petioles have to be stripped off causing the celery to become smaller and the functional yields to be decreased.

Texture rating. Means the feel of the celery in the mouth of a human or the malleability of the celery.

Vacuum. Means the negative pressure (in inches mercury) required to rupture or break the celery petiole.

Vascular Bundle. Means the xylem and phloem run vertically through the petiole near the epidermis in groups or traces called vascular bundles.

Vegetable material. Means products that are derived from, but not limited to, vegetables, fruit, grains and other plants.

### DETAILED DESCRIPTION OF THE INVENTION

The following embodiments and aspects thereof are described in conjunction with systems, tools, and methods which are meant to be exemplary and illustrative, not limiting in scope. In various embodiments, one or more of the above-described problems have been reduced or eliminated, while other embodiments are directed to other improvements.

Drinking straws are so named because they were originally cut from hollow wheat straw. Over the years, paper and plastics became the preferred material for drinking straws. Nevertheless, it would be advantageous to have an edible and natural material which could be used both for drinking straws, thus forming an edible element of the product being served, and as a holder or container for another foodstuff.

Celery has been developed into three types with different uses.

Stalk celery, Apium graveolens L. var. dulce, is the most prevalent in the United States and Western Europe. This celery has been developed to have large, fairly flat to cupped stems with a solid, crisp interior. Frequently, the top-most leaves and roots are removed before sale and consumption. Stems with entire leaves are available for consumption in the center of the plant, called the heart, and these stems are sold like outer stems. Breeding has been used to make stalk celery greener, of milder flavor and generally more solid and crisp.

Previous to the present invention, there are no known Apium graveolens L. var. dulce varieties having a hollow petiole. It is a feature of the present invention that the hollow petioles trait in Apium graveolens L. var. dulce of the present invention has been and can be used in and transferred among other Apium graveolens L. var. dulce varieties.

Root celery or celeriac, Apium graveolens L. var. rapaceum is often grown in cooler climates. Unlike stem celery, root celery has been developed to have an enlarged hypocotyl or root bulb often weighing one to two pounds. The stems, leaves and small roots are removed from this swollen root ball, which is used much like the potato, and which may be fried or mashed, or used in stews and soups. The stems and leaves are more bitter and more fibrous than those of stem celery. The stems may be hollow, solid, or in between.

Leaf celery or smallage, Apium graveolens L. var. secalinum has been developed primarily for leaf and seed production. Often grown in Mediterranean climates, leaf celery more closely resembles celery's wild ancestors. The stems are small and fragile and vary from solid to hollow and the leaves are fairly small and are generally bitter. This type is often used for its medicinal properties and spice.

The present invention encompasses an Apium graveolens L. var. dulce hollow celery stick having an average wall thickness at the inside of the petiole cup between 0.67 mm, 0.73 mm, 0.85 mm, 0.93 mm, 1.11 mm, 1.23 mm, 1.35 mm, 1.65 mm, 1.75 mm, 1.84 mm, 1.96 mm, 2.02 mm, 2.13 mm, 2.24 mm, 2.52 mm, 2.66 mm, 2.75 mm and 2.89 mm or higher and including all integers and fractions thereof.

The present invention encompasses an Apium graveolens L. var. dulce hollow celery stick that is capable of withstanding a vacuum pressure between 10.4 in/Hg, 11.1 in/Hg, 11.9 in/Hg, 12.1 in/Hg, 13.3 in/Hg, 14.6 in/Hg, 15.1 in/Hg, 15.9 in/Hg, 16.1 in/Hg, 18.6 in/Hg, 19.1 in/Hg, 19.9 in/Hg, 22.1 in/Hg, 24.0 in/Hg, 25.5 in/Hg, 26.3 in/Hg, 28.0 in/Hg and 29.6 in/Hg or higher and including all integers and fractions thereof.

The present invention encompasses an Apium graveolens L. var. dulce hollow celery stick having an average width of outer petiole between 7.5 mm, 8.4 mm, 9.6 mm, 10.2 mm, 11.7 mm, 12.2 mm, 12.9 mm, 13.7 mm, 14.5 mm, 16.2 mm, 17.4 mm, 18.9 mm, 19.8 mm, 23.2 mm or higher and including all integers and fractions thereof.

The present invention encompasses an Apium graveolens L. var. dulce hollow celery stick having an average depth of outer petiole between 6.2 mm, 7.2 mm, 8.4 mm, 9.6 mm, 10.2 mm, 11.7 mm, 12.2 mm, 12.9 mm, 13.7 mm, 14.5 mm, 16.2 mm, 17.4 mm, 18.9 mm and 19.2 mm or higher and including all integers and fractions thereof.

The present invention encompasses an Apium graveolens L. var. dulce hollow celery stick having an average depth of outer petiole between 7.2 mm, 8.4 mm, 9.6 mm, 10.2 mm, 11.7 mm, 12.2 mm, 12.9 mm, 13.7 mm, 14.5 mm, 16.2 mm, 17.4 mm, 18.9 mm and 19.2 mm or higher and including all integers and fractions thereof.

The present invention encompasses an Apium graveolens L. var dulce hollow celery with a wall thickness at the inside petiole cup tissue between 0.67 mm and 2.89 mm or higher and including all integers and fractions thereof.

The present invention encompasses an Apium graveolens L. var dulce hollow celery with a wall thickness at the sidewall of the petiole between 1.50 mm, 1.68 mm, 2.33 mm, 2.87 mm, 3.05 mm, 3.46 mm, 3.89 mm, 4.02 mm, 4.44 mm, and 5.00 mm or higher and including all integers and fractions thereof.

The present invention encompasses an Apium graveolens L. var dulce hollow celery plant that has an inside petiole cup tissue with the ability to withstand pressure between 300 g, 350 g, 431 g, 446 g, 523 g, 638 g, 811 g, 966 g, 1052 g, 1189 g, and 1300 grams of pressure or higher and including all integers and fractions thereof.

### Example 1

### Flavor of the Hollow Celery Stick

The development of a new class of edible hollow celery stick was initiated in order to provide an edible product that would be functional as celery straws and food stuffed hollow celery products. Until a suitable edible hollow celery stick variety was developed, no final product development could occur for straws or stuffed products.

The present invention is developed from, or utilize edible hollow celery stick as a primary ingredient may differ in their specifications of the hollow celery stick that is required in the product/process.

The present invention differs from celeriac and root celery products that are currently available in that the hollow petioles of the celery of the present invention have a thickened and succulent leaf petiole that has a mild taste, whereas the hollow-stick or petiole of celeriac and root celery (Apium graveolens L. var. rapaceum) such as the celeriac variety PI 179171 have enlarged hypocotyls and upper roots with a hollow petiole that is extremely bitter and fibrous. Unlike the hollow leaf petioles of celeriac and root celery which usually split and rupture starting at their base or connection point with the basal plate and move upward the petiole causing the petiole to be unusable as a straw those of the proposed new product remain intact. These splits or petiole ruptures occur in celeriac as the root expands or enlarges during growth.

The use of Apium graveolens L. var. secalinum or leaf celery varieties is also inappropriate for the products of the present inventions. Leaf celery, typically harvested for the leaves or utilized to produce seed for spice may or may not have hollow stems, but those with hollow stems are also unsuitable. The stems of Apium graveolens L. var. secalinum are too small in diameter, very tender and will not withstand the vacuum. Apium graveolens L. var. secalinum celery does not have primary petioles like Apium graveolens L. var. dulce celery but is primarily a stalk consisting primarily of small sucker petioles. Apium graveolens L. var. secalinum does not produce a single primary growing point on the basal plate with a single whorl of petioles developing around it like the traditional stalk from stalk celery. Instead Apium graveolens L. var. secalinum has little to no apical dominance and several growing points develop that produce an abundance of miniature stalks, so to speak, on the basal plate. These small petioles are tender, very thin and fibrous. Leaf celery also is very bitter in flavor.

It is possible that in a single product, the specifications may be different for different customers, or end uses/users. Thus, different varieties may be refined within the class of edible, hollow celery stick to meet the specific criteria, or specifications for the individual product.

For example, some of the products or customers require edible hollow-sticks with larger diameters and others require edible hollow-sticks with smaller diameters.

Flavor in celery is a complex of several compounds which together create a flavor profile for a variety. Several different classes of compounds act together at varying levels to create a flavor that is not only unique for celery, but fairly unique to the individual variety. Some of these compound groups include, but are not limited to sugars, phthalides, carotenoids, linear furanocoumarins, etc.

There are three primary types of sugar that maybe actively involved in the flavor profile for celery and each has its own characteristic contribution to the overall sweetness of the celery. For instance fructose which is commonly found in celery has a sweetness equivalent of 140 (Relative Sweetness Scale) while glucose has a sweetness rating of 70 to 80 and sucrose has a rating of 100. Each variety may have a different ratio of each of these sugars, hence a different sweetness contribution

Similarly, there are several different phthalides, carotenoids and furanocumarins that may contribute to the overall flavor complex in celery with each making a slightly different contribution. Carotenoids are frequently associated with the carrot flavor of carrots and similarly have a little contribution to the overall flavor of celery.

However, the most prominent set of compounds that have the single most dramatic effect on the flavor of celery belong to two classes; the phalides which are responsible for the prominent characteristic celery flavor, and the furanocoumarins which affect t the bitterness of the celery. The furanocoumarin class encompasses three particular compounds in celery belong, psoralen, bergapten and xanthotoxin. This set of compounds is essentially responsible for the strong, slightly bitter flavor associated with celery. In fact higher levels of furanocoumarin type compounds frequently mask the flavor contributed by the sugars and carotenoids in celery. These same compounds are responsible for natural plant defense responses in celery and become elevated when celery is diseased or grown under stressful conditions. Furanocoumarins are also responsible for the phenomenon called celery rash which may occasionally affect handlers of celery. When present on a person's skin, furanocoumarins may be photo-activated by light and cause a rash similar to poison ivy in susceptible persons. The levels of furanocoumarins are generally highest in the wild species of celery, celeriacs (also known as root celery, are related to celery but instead of having a thickened and succulent leaf petiole as in celery, celeriacs have an enlarged hypocotyl and upper root that is the edible product) and leaf celery.

This flavor complex can vary for a variety from one production condition to another, especially under conditions that vary for the presence of disease and/or stress. However, the relationship of flavor between varieties remains fairly constant with respect to one another under these varying conditions. Therefore a table rating the overall flavor of different varieties in relationship to one another is an excellent means for comparing the overall flavor for an individual variety.

Table 1 shows the flavor rating of several varieties. The flavor rating is based on the mouth flavor and taste of each individual celery variety. The varieties tested were grown concurrently and evaluated side by side. The flavor rating ranges from 1 (sweet) to 10 (bitter). Most common cup-stem celery varieties are in a range from 3 to 5 which is generally considered mild flavor. Celery leaf and celery root varieties are classified 9 and 10 (bitter).

TABLE 1
Flavor Ratings (1-10)

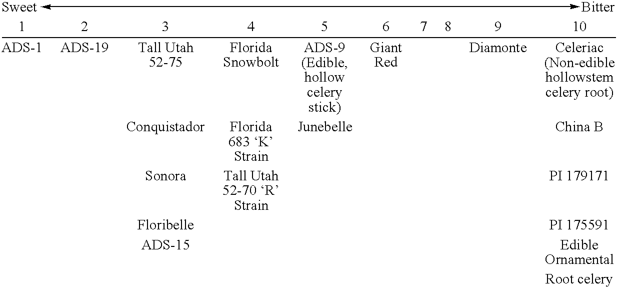

Root celery are commercial variety names as opposed to the general type or classification of celery referred to as celeriac or root celery.

Lines that are prefaced with PI (Plant Introductions) are items that have been donated, found, or otherwise collected as wild types, finished varieties, old land races, etc. and are now part of the USDA-ARS Plant Germplasm Resource system. These lines are entered into the USDA-ARS system and assigned Plant Introduction numbers (PI).

### Example 2

### Texture of the Hollow Celery Stick

The fibrous character in celery is a reflection of the types and nature of the cells that the tissues of the celery are composed of. A cross section of the celery petiole of a conventional stem celery variety is characterized by a thin layer of epidermal and chollenchyma cells at the surface and a layer of palisade mesophyll cells just below the surface. These cells are characterized by smaller cells having less vacuoles storing water and the region has a greater level of fibrous cell wall material. It is also reinforced with a higher percentage of thin parenchyma cells containing cellulose microfibrils which provide reinforcement and affect the fibrous nature. This region of the celery also has fairly rigid vascular bundles running vertically through the stem. The tissue below these cells and constituting the center of the petiole is composed of spongy mesophyll cells which contain large vacuoles filled with water, air and other metabolic substances. In some celery varieties the spongy cells in this region are less densely packed so there is a greater amount of intercellular space. These spongy mesophyll cells are much less fibrous with the cell wall to fluid ratio being considerably decreased. In varieties that pith easily there is much more intercellular space and the cells present readily give up their moisture at or near maturity and collapse. This area becomes filled with air and becomes pithy.

The difficulty with a hollow petiole is a majority of these less fibrous spongy mesophyll cells are absent producing a hollow petiole. Root and leaf celery varieties are essentially devoid of spongy mesophyll cells and the stem is composed almost entirely of palisade, parenchyma, chollencyma and epidermal cells. As a result these varieties are extremely fibrous. This is reflected by higher percentage dry weight which is calculated by the amount of solids divided by solids and liquids. In the development of the hollow petiole stem celery varieties of the present invention such as ADS-19 special attention is made to retain more of the spongy mesophyll cells characteristic to the stem celery parent. Varieties like ADS-19 of the present invention which have a thicker wall around the hollow tube and a significant layer of spongy mesophyll cells have a lower percentage dry weight. Since percentage dry weight reflects this relationship or ratio of spongy mesophyll to parenchyma, chollenchyma, pallisade and epidermal cells and it is accordingly a good indicator for fibrousness. A higher percentage of parenchyma, chollenchyma, pallisade and epidermal cells generally produces a higher percentage of dry weight and respectively a greater fibrousness.

The level of fibrousness in celery, like flavor, is often affected by environmental conditions with different levels of stress, drought, maturity, fertility and disease having an effect. However, the relationship of texture between varieties remains fairly constant with respect to one another under these varying conditions. Therefore a table rating the overall texture of different varieties in relationship to one another is an excellent means for comparing the overall fibrousness for an individual variety.

Table 2 shows the texture rating for several varieties. The texture ratings is based on the mouth feel or malleability of each individual celery variety. The varieties tested were grown concurrently and evaluated side by side. The texture ratings range from 1 (non-fibrous) to 10 (fibrous). Most stem celery varieties range from 2 to 6 which is generally considered less fibrous. Celery leaf and celery root varieties are generally classified as 9 to 10 (fibrous). Hollow celery stick varieties are rated 1 to 6.

TABLE 2
Texture Rating (1-10)

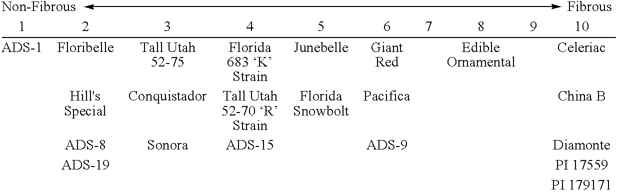

Root celery are commercial variety names as opposed to the general type or classification of celery referred to as celeriac or root celery.

Lines that are prefaced with PI are items that have been donated, found, or otherwise collected as wild types, finished varieties, old land races, etc. and are now part of the USDA-ARS Plant Germplasm Resource system. These lines are entered into the USDA-ARS system and assigned Plant Introduction numbers (PI).

The present invention can be used as a straw for beverages such as tomato juice, Bloody Mary drinks and beer. The advantage of using the hollow celery stick as a straw is that the straw adds natural celery flavor to the beverage as it is consumed through the straw; the acids of the tomato, vegetable or fruit and the alcohol, extract the natural celery flavor from the celery stick as the beverage passes through the straw. This celery flavor acts to enhance the overall beverage character. The natural celery straw can then be consumed once the drink is completed. If the celery straw is not consumed, unlike commercial plastic straws, the celery straw has an advantage of being totally biodegradable.

The diameter of the hollow celery stick required for the straw may vary, but it will generally be, but not limited to, 0.7 cm to 1.25 cm in diameter; this is primarily due to the difficulty in drinking a beverage comfortably through a very large straw.

The length of the straw itself may also vary depending on the size of the glass used or the customers' individual preferences. Standard plastic straws are generally 17.8 cm to 20.3 cm, but can be found up to 28.0 cm to 30.5 cm. Celery straws can be produced and cut to meet the same measurements.

Some drinks, especially those that are more viscous may require larger diameter straws in order to get the beverage to flow.

The present invention of food stuffed hollow celery stick may take several forms. Stuffed, raw celery sticks may require hollow celery sticks with different dimensions depending on the specifications, product format or presentation.

Stuffed, raw hollow celery sticks of the present invention are edible celery that are cut to a particular length and stuffed or filled with a product to enhance the celery. These stuffing or filling products can include but are not limited to dairy based products, synthetic food types, nut based fillings, soy based products, chocolate, fruits and vegetable products, candy products, ethnic flavorings such as products with Mexican, Japanese, Chinese or Indian flavors, fillings with preservatives, amendments to modify textures such as starches or to control moisture levels, products with nutritional fortification including but not limited to minerals such as calcium and potassium, vitamins including but not limited to A, B1 (thiamine), B2 (Riboflavin), B6 (Niacin), B12 (biotin, folic acid and cyanocobalamin) C, and D, E and K as well as minerals including but not limited to calcium, potassium, chromium, copper, manganese, selenium, and zinc.

The fillings may have to be specially formulated to be injected into the hollow celery sticks, but can be of essentially any flavor. Currently most of these food products are spread over the surface of the raw celery stick and tend to be messy. Stuffed celery will be less messy and may be classified as ready to eat on the go food.

The diameter of the raw, stuffed celery of the present invention is anticipated to be, but is not limited to, medium to large diameter hollow celery sticks approximately 0.938 to 1.875 cm in diameter. Again, different customers may have different specifications (length, diameter, thickness, color, etc.) for their particular use.

Some of these specifications may change as the products develop or as new uses evolve so corresponding refinements may have to be made to either the hollow celery stick varieties or the process in which they are cut and prepared.

Cooked stuffed hollow celery sticks may be similar to raw stuffed hollow celery sticks except it is cooked in its final form.

The product of the present invention may take various forms including, but not limited to, being filled with a cheese type product and baked like a manicotti, or filled and cooked like an enchilada, or stuffed, battered and deep fried like a jalapeno popper.

Some of these products may be precooked and frozen, others may be stuffed and frozen in the raw state for cooking later and still others may be stuffed in the raw state and sold fresh for cooking.

This type of product is likely to utilize larger diameter hollow-stick celery varieties (1.25 to 5.1 cm in diameter) but the specifications may vary by product or customer preference.

### Tables

In the tables that follow, the traits and characteristics of the present invention, including celery cultivars ADS-19, ADS-15 and ADS-9 are given compared to other publicly available cultivars.

Table 3 shows a comparison between ADS-15, ADS-19 and ADS-9 of the present invention in a trial grown in Oxnard, Calif. The trial was transplanted Mar. 6, 2007 at a population of 58,080 plants per acre. Production conditions were typical with out significant stress.

TABLE 3

| | ADS-15 | ADS-19 | ADS-9 |
|---|---|---|---|
| | Apium | Apium | Apium |
| | graveolens | graveolens | graveolens |
| | L. var | L. var | L. var |
| | dulce | dulce | dulce |
| Maturity (days) | 84 | 87 | 87 |
| Mean Plant Height (cm) | 87.5 | 87.3 | 85.9 |
| Mean Whole Plant | 0.728 | 0.798 | 0.513 |
| weight (kg) | | | |
| Mean Trim Plant weight | 0.605 | 0.739 | 0.494 |
| (kg) | | | |
| Mean Number of | 0 | 0 | 15.6 |
| Suckers | | | |
| Mean Joint Length (cm) | 47.6 | 44.2 | 50.1 |
| Mean Number of Outer | 10.6 | 10.4 | 14.2 |
| Petioles >40 cm | | | |
| Mean Number of Inner | 4 | 4 | 4.7 |
| Petioles <40 cm | | | |
| Mean Width of Outer | 15.7 | 17.9 | 10.4 |
| Petioles @ midrib (mm) | | | |
| Mean Depth of Outer | 12 | 12.3 | 7.1 |
| Petioles @ midrib (mm) | | | |
| Mean Wall Thickness at | 3.7 | 4.5 | 2 |
| Sides of Petiole (mm) | | | |
| Mean Number of 7 inch | 14.9 | 13.1 | 22 |
| Straws per Plant | | | |
| Mean Straw Yield per | 0.264 | 0.295 | 0.233 |
| Plant (kg) | | | |
| Mean Weight per Straw | 17.7 | 22.5 | 10.6 |
| (g) | | | |
| Number of 7 inch Straws | 865,392 | 760,848 | 1,277,760 |
| per Acre | | | |
| Straw Yield per Acre | 15,333 | 17,134 | 13,533 |
| (kg) | | | |

As shown in Table 3, ADS-15 of the present invention unexpectedly produces a stalk that looks very similar to a compact, well shingled conventional celery with large petioles. If it were not for the fact that the limbs are hollow it would be easily mistaken for a conventional type celery with a taller joint. The only characteristic it possesses from its celeriac progentor parent are hollow stems. This contrasts with ADS-9 of the present invention which unexpectedly has characteristics intermediate between its leaf celery progenitor and its conventional progenitor, in particular a greater quantity of smaller petioles, and a few suckers. Leaf celery has no prominent whorl of petioles and is essentially all suckers. ADS-15 unexpectedly had 25% fewer stems that were approximately 50% wider, 69% thicker, had side walls approximately 85% thicker than ADS-9 and ADS-19. It produced 30% fewer 7-inch straws per plant but the straws produced were approximately 13% heavier than ADS-9 and ADS-19.

Compared to ADS-15, ADS-19 of the present invention is unexpectedly slightly shorter to the joint, produces slightly fewer petioles, and weighs approximately 10% more in the whole stalk. ADS-19's main points of differentiation are a wider petiole (14%) that is 22% thicker and gives the finished straw yield per plant and acre 12% more weight in spite of yielding 14% fewer straws than ADS-15. This wider, thicker petiole in ADS-19 is more conducive and desirable for stuffing while the ADS-15 is preferred for straws for drinking beverages.

Table 4 shows a comparison between ADS-15, ADS-19 and ADS-9 of the present invention in a trial grown in Oxnard, Calif. The trial was transplanted Mar. 13, 2007 at a population of 58,080 plants per acre. Production conditions were typical with out significant stress.

TABLE 4

| | ADS-15 | ADS-19 | ADS-9 |
|---|---|---|---|
| | Apium | Apium | Apium |
| | graveolens | graveolens | graveolens |
| | L. var dulce | L. var dulce | L. var dulce |
| Maturity (days) | 80 | 87 | 87 |
| Mean Plant Height | 91.8 | 87 | 83 |
| (cm) | | | |
| Mean Whole Plant | 0.731 | 0.801 | 0.486 |
| weight (kg) | | | |
| Mean Trim Plant | 0.599 | 0.712 | 0.453 |
| weight (kg) | | | |
| Mean Number of | 0 | 0 | 18.2 |
| Suckers | | | |
| Mean Joint | 46.2 | 44.2 | 47.8 |
| Length(cm) | | | |
| Mean Number of | 10.3 | 9.8 | 13.3 |
| Outer Petioles >40 cm | | | |
| Mean Number of | 3.1 | 2.9 | 4.6 |
| Inner Petioles <40 cm | | | |
| Mean Width of Outer | 16.2 | 18.3 | 11.1 |
| Petioles @ midrib | | | |
| (mm) | | | |
| Mean Depth of Outer | 12.0 | 12.5 | 7.0 |
| Petioles @ midrib | | | |
| (mm) | | | |
| Mean Wall Thickness | 3.7 | 4.6 | 2.6 |
| at Sides of Petiole | | | |
| (mm) | | | |
| Mean Number of 7 | 12.9 | 11.7 | 20.2 |
| inch Straws per Plant | | | |
| Mean Straw Yield per | 0.255 | 0.265 | 0.206 |
| Plant (kg) | | | |
| Mean Weight per | 19.8 | 22.6 | 10.2 |
| Straw (g) | | | |
| Number of 7 inch | 749,232 | 679,536 | 1,173,216 |
| Straws per Acre | | | |
| Straw Yield per Acre | 14,810 | 15,391 | 11,964 |
| (kg) | | | |

As shown in Table 4, ADS-15 unexpectedly produces a stalk that looks very similar to a compact well shingled conventional celery with large petioles. If it were not for the fact that the limbs are hollow it would be easily mistaken for conventional type celery with a taller joint. The only characteristic it possesses from its celeriac progenitor parent are hollow stems. This contrasts with ADS-9 which unexpectedly has characteristics intermediate between its leaf celery progenitor and its conventional progenitor, in particular a greater quantity of smaller petioles, and a few suckers. Leaf celery has no prominent whorl of petioles and is essentially all suckers. ADS-15 had 30% fewer stems that were approximately 46% wider, 71% thicker and had side walls approximately 85% thicker than ADS-19 and ADS-9. It produced 36% fewer 7-inch straws per plant but the straws produced were approximately 24% heavier.

Compared to ADS-15, ADS-19 is slightly shorter to the joint, produces slightly fewer petioles and weighs approximately 10% more in the whole stalk. ADS-19's main points of differentiation are a wider petiole (13%) that is 25% thicker and gives the finished straw yield per plant and acre 4% more weight in spite of yielding 10% fewer straws than ADS-15. This wider thicker petiole in ADS-19 is more conducive and desirable for stuffing while the ADS-15 is preferred for straws for drinking beverages.

Table 5 shows a comparison between ADS-19, ADS-9 and ADS-15 of the present invention, Blanco de Veneto (Apium graveolens L. var rapaceum) a celeriac or root celery and Afina (Apium graveolens L. var secalinum) a leaf celery in a trial grown in Oxnard, Calif. The trial was transplanted December 2007 at a population of 58,080 plants per acre. Production was during a period when bolting pressure was severe. Harvest occurred Apr. 21, 2008 at 115 days maturity.

TABLE 5

| | ADS-15 | ADS-9 | ADS-19 | Blanco de Veneto | Afina |
|---|---|---|---|---|---|
| | Apium graveolens | Apium graveolens | Apium graveolens | Apium graveolens | Apium graveolens |
| | L. var dulce | L. var dulce | L. var dulce | L. var rapaceum | L. var secalinum |
| Mean Plant Height (cm) | 95.1 | 96.7 | 100.6 | 73.2 | 80.7 |
| Mean Whole Plant weight | 0.865 | 0.814 | 0.911 | 0.497 | 0.744 |
| (kg) | | | | | |
| Mean Root Diam. (cm) | 0.0 | 0.0 | 0.0 | 51.8 | 0.0 |
| Mean Root weight (kg) | 0.0 | 0.0 | 0.0 | 0.072 | 0.0 |
| Mean Joint Length (cm) | 49.3 | 57.2 | 52.6 | 29.7 | 45.4 |
| Mean Number of Outer | 8.3 | 11.9 | 8.1 | 6.8 | 22.6 |
| Petioles >40 cm | | | | | |
| Mean Number of Inner | 5.1 | 8.9 | 5.0 | 7.3 | 85.2 |
| Petioles <40 cm | | | | | |
| Mean Number of Suckers | 0.0 | 17.0 | 0.0 | 2.0 | 101.0 |
| Mean Seed Stem Length | 8.8 | 45.7 | 9.9 | 35.9 | 23.3 |
| (cm) | | | | | |
| Mean Width of Outer | 17.7 | 11.7 | 18.4 | 7.3 | 6.0 |
| Petioles @ midrib (mm) | | | | | |
| Mean Depth of Outer | 16.6 | 11.3 | 16.9 | 6.9 | 5.2 |
| Petioles @ midrib (mm) | | | | | |
| Mean Vacuum (in/Hg) | 22.3 | 25.9 | 21.1 | 10.1 | 11.5 |
| Mean Wall Thickness at | 4.2 | 2.8 | 4.5 | 2.2 | 1.4 |
| Sides of Petiole (mm) | | | | | |
| Mean Wall Thickness at | 1.2 | 1.0 | 1.5 | 0.6 | 0.6 |
| Inside of Petiole Cup | | | | | |
| (mm) | | | | | |
| Mean Pressure Required | 2257.2 | 1198.0 | 1886.8 | 817.5 | 505.8 |
| to Rupture Side | | | | | |
| Wall (grams) | | | | | |
| Mean Pressure Required | 343.4 | 398.7 | 325.3 | 149.4 | 164.3 |
| to Rupture Wall @ Inside | | | | | |
| of Petiole Cup (grams) | | | | | |
| Leaf Color (Muncell) | 5gy 4/6 | 5gy 4/8 | 5gy 4/6 | 5gy 4/4 | 5gy 4/6 |
| Petiole Color (Muncell) | 5gy 6/6 | 5gy 6/6 | 5gy 6/8 | 5gy 5/6 | 5gy 5/10 |
| Petiole Smoothness | slight rib | slight rib | smooth | ribbed | ribbed |

As shown in Table 5, ADS-15 and ADS-19 are the most similar in these results and unexpectedly have a significantly thicker and stronger petiole side wall and petiole cup than Blanco de Veneto (Apium graveolens L. var rapaceum) a celeriac or root celery and Afina (Apium graveolens L. var secalinum) a leaf celery. While ADS-15 is designed for use as a straw, ADS-19 is unexpectedly a larger, less fibrous celery line designed for stuffing and use for actual consumption as opposed to a straw. The ADS-19 and ADS-15 varieties (Apium graveolens L. var dulce) are contrasted to Blanco de Veneto (Apium graveolens L. var rapaceum) a celeriac or root celery and Afina (Apium graveolens L. var secalinum) a leaf celery. The Blanco de Veneto measurements for root diameter and depth are included but do not represent a fully mature root; however the petioles are at full size and represent a reasonable comparison to ADS-15 and ADS-19. ADS-15 and ADS-19 unexpectedly, are fairly bolting tolerant and significantly more bolting tolerant than the celeriac and leaf celery varieties. ADS-15 unexpectedly has a significantly wider and deeper petiole, thicker side wall and inside of cup wall, requires significantly more pressure to rupture the side wall of the petiole and inside of cup of the petiole when compared to ADS-9 and Blanco de Veneto and Afina. The ability to withstand a vacuum was also significantly improved when compared to Blanco de Veneto and Afina, however the ability to withstand vacuum pressure by ADS-19 was less than ADS-9. ADS-15 was not as thick at the petiole side wall and inside of the cup as ADS-19 but unexpectedly still significantly thicker than Blanco de Veneto (Apium graveolens L. var rapaceum) a celeriac or root celery and Afina (Apium graveolens L. var secalinum) a leaf celery. ADS-15 and ADS-19 unexpectedly have no suckers when compared with ADS-9 and Afina. Afina which is essentially a stalk comprised primarily of suckers is very typical of leaf celery while ADS-9 has only a few suckers characteristic of its leaf celery progenitor and is more similar to a stem celery with a higher sucker count. This contrasts to ADS-15 and ADS-19 which have a celeriac as one of the originating parents. While celeriacs may vary in the number of suckers possessed, dependent on the variety, ADS-15 and ADS-19 are more similar to tall stem varieties like ADS-11, ADS-17 and ADS-18 which typically have no suckers.

Table 6 shows a comparison between ADS-19 of the present invention, Afina (Apium graveolens L. var secalinum) and numerous hollow-stem celeriacs (Apium graveolens L. var rapaceum), including commercial and representatives from the United States germplasm collection. All comparisons were generated from a trial harvested May 29, 2008 in Oxnard, Calif. The trial was transplanted Feb. 27, 2008 at a population of 58,080 plants per acre. Production was during a period when conditions were fairly normal and free from most stresses. This data shows that there may have been marginal bolting pressure, but pressure was light and the presence of seed stems is an indication that the varieties were particularly sensitive. Harvest occurred May 29, 2008 at 92 days maturity.

TABLE 6

| | | Blanco de | | | | |
|---|---|---|---|---|---|---|
| | ADS-19 | Veneto | Monarch | Afina | PI 193454 | PI 179171 |
| | Apium | Apium | Apium | Apium | Apium | Apium |
| | graveolens | graveolens L. var | graveolens L. var | graveolens L. var | graveolens L. var | graveolens L. var |
| | L. var dulce | rapaceum | rapaceum | secalinum | rapaceum | rapaceum |
| Mean Plant Height (cm) | 91.4 | 49.3 | 45.9 | 55.6 | 53.9 | 67.9 |
| Mean Plant Width (cm) | 34.8 | 36.1 | 27.7 | 41.6 | 44.4 | 45.0 |
| Mean Whole Plant weight | 0.967 | 0.368 | 0.301 | 0.531 | 0.409 | 0.465 |
| (kg) | | | | | | |
| Mean Trimmed Plant | 0.901 | 0.3295 | 0.1935 | 0.198 | 0.295 | 0.249 |
| Weight (kg) | | | | | | |
| Mean Number of Suckers | 0.0 | 0.0 | 0.0 | 103.8 | 0.0 | 25.8 |
| Mean Root Diameter (cm) | 0.0 | 56.8 | 61.9 | 0.0 | 82.0 | 66.0 |
| Mean Root Depth (cm) | 0.0 | 64.2 | 76.1 | 0.0 | 96.0 | 80.0 |
| Mean Root weight (kg) | 0.0 | 0.114 | 0.115 | 0.0 | 0.258 | 0.213 |
| Mean Joint Length (cm) | 44.6 | 20.0 | 17.8 | 24.8 | 27.8 | 37.6 |
| Mean Number of Outer | 10.0 | 9.7 | 12.1 | 7.4 | 12.6 | 11.9 |
| Petioles (>40 cm) | | | | | | |
| Mean Number of Inner | 6.6 | 9.8 | 6.5 | 9.2 | 7.1 | 5.0 |
| Petioles (<40 cm) | | | | | | |
| Mean Seed Stem Length | 0.0 | 0.0 | 0.0 | 0.0 | 0.5 | 1.2 |
| (cm) | | | | | | |
| Mean Width of Outer | 18.1 | 7.3 | 7.1 | 6 | 7.5 | 7.5 |
| Petioles @ midrib (mm) | | | | | | |
| Mean Depth of Outer | 11.9 | 6.5 | 6.5 | 3.9 | 4.6 | 6.6 |
| Petioles @ midrib (mm) | | | | | | |
| Mean Number of 7 inch | 12.4 | 1.1 | 0 | 7 | 7.6 | 11.7 |
| Straws per Plant | | | | | | |
| Mean Straw Yield per | 0.320 | 0.012 | 0 | 0.044 | 0.061 | 0.084 |
| Plant (kg) | | | | | | |
| Mean Weight per Straw | 25.8 | 10.5 | 0 | 6.3 | 8 | 7.2 |
| (g) | | | | | | |
| Number 7″ Straws per | 720,192 | 63,888 | 0 | 406,560 | 441,408 | 679,536 |
| Acre | | | | | | |
| Straw Yield per Acre (kg) | 18,586 | 668 | 0 | 2,556 | 3,543 | 4,879 |
| Mean Vacuum (in/Hg) | 25.3 | 10.7 | 11.2 | 11.5 | 12.9 | 14.0 |
| Mean Wall Thickness at | 4.18 | 1.82 | 2.07 | 1.26 | 1.01 | 1.42 |
| Sides of Petiole (mm) | | | | | | |
| Mean Wall Thickness at | 1.44 | 0.75 | 0.55 | 0.39 | 0.54 | 0.37 |
| Inside of Petiole Cup | | | | | | |
| (mm) | | | | | | |
| Mean Pressure Required | 2105 | 1554 | 1630 | 1055 | 990 | 698 |
| to Rupture Side Wall (g) | | | | | | |
| Mean Pressure Required | 661 | 417 | 444 | 414 | 292 | 145 |
| to Rupture Wall @ Inside | | | | | | |
| Of Petiole Cup (g) | | | | | | |
| Mean Percentage Dry | 6.2% | 8.0% | 7.7% | 8.5% | 8.2% | 9.2% |
| Weight | | | | | | |

As shown in table 6, ADS-19 is unexpectedly 10% to 21% taller than the hollow-stem leaf celery and celeriac lines. ADS-19 does not have suckers. ADS-19 also unexpectedly has a significantly thicker petiole side wall and petiole cup than the Apium graveolens L. var secalinum and Apium graveolens L. var rapaceum varieties. While Afina is a typical representative of the leaf celery class which is essentially all suckers, the celeriacs have a fairly wide range of suckers from 0 to 50 per stalk. None of the celeriac varieties have the preponderance of suckers as represented by Afina. An unexpected significant difference between ADS-19 and the root celery lines (Apium graveolens L. var rapaceum) is the absence of a swollen root (tuber). While the maturity was not sufficient to allow for maximum swelling/yield there was obvious and measureable swelling in the celeriacs. Measurements were taken for width, depth and weight. No swelling had occurred in ADS-19 stem celery (Apium graveolens L. var dulce) or leaf celery (Apium graveolens L. var secalinum). Differences between ADS-19 when compared with all other classes became especially pronounced when the petiole width and thickness was measured at the mid-rib. The total number of straws or 7-inch petiole segments varied among all of the lines but when all characteristics, including width thickness, vacuum, rupture pressure and wall thickness were considered only ADS-19 made reasonably usable straws. When comparing wall thickness, vacuum and rupture pressure ADS-19 was unexpectedly much more durable than the celeriac and leaf celery lines.

Table 7 shows a comparison between ADS-15 of the present invention, Afina (Apium graveolens L. var secalinum) and numerous hollow-stem celeriacs (Apium graveolens L. var rapaceum), including commercial and representatives from the United States germplasm collection. All comparisons were generated from a trial harvested May 29, 2008 in Oxnard, Calif. The trial was transplanted Feb. 27, 2008 at a population of 58,080 plants per acre. Production was during a period when conditions were fairly normal and free from most stresses. This data shows that there may have been marginal bolting pressure, but pressure was light and the presence of seed stems is an indication that the varieties were particularly sensitive. Harvest occurred May 29, 2008 at 92 days maturity.

TABLE 7

| | | Blanco de | | | | |
|---|---|---|---|---|---|---|
| | ADS-15 | Veneto | Monarch | Afina | PI 193454 | PI 179171 |
| | Apium | Apium | Apium | Apium | Apium | Apium |
| | graveolens | graveolens L. var | graveolens L. var | graveolens L. var | graveolens L. var | graveolens L. var |
| | L. var dulce | rapaceum | rapaceum | secalinum | rapaceum | rapaceum |
| Mean Plant Height (cm) | 98.7 | 49.3 | 45.9 | 55.6 | 53.9 | 67.9 |
| Mean Plant Width (cm) | 33.7 | 36.1 | 27.7 | 41.6 | 44.4 | 45.0 |
| Mean Whole Plant weight | 0.851 | 0.368 | 0.301 | 0.531 | 0.409 | 0.465 |
| (kg) | | | | | | |
| Mean Trimmed Plant | 0.673 | 0.3295 | 0.1935 | 0.198 | 0.295 | 0.249 |
| Weight (kg) | | | | | | |
| Mean Number of Suckers | 0.0 | 0.0 | 0.0 | 103.8 | 0.0 | 25.8 |
| Mean Root Diameter (cm) | 0.0 | 56.8 | 61.9 | 0.0 | 82.0 | 66.0 |
| Mean Root Depth (cm) | 0.0 | 64.2 | 76.1 | 0.0 | 96.0 | 80.0 |
| Mean Root weight (kg) | 0.0 | 0.114 | 0.115 | 0.0 | 0.258 | 0.213 |
| Mean Joint Length (cm) | 49.4 | 20.0 | 17.8 | 24.8 | 27.8 | 37.6 |
| Mean Number of Outer | 9.6 | 9.7 | 12.1 | 7.4 | 12.6 | 11.9 |
| Petioles (>40 cm) | | | | | | |
| Mean Number of Inner | 5.6 | 9.8 | 6.5 | 9.2 | 7.1 | 5.0 |
| Petioles (<40 cm) | | | | | | |
| Mean Seed Stem Length | 0.0 | 0.0 | 0.0 | 0.0 | 0.5 | 1.2 |
| (cm) | | | | | | |
| Mean Width of Outer | 16.9 | 7.3 | 7.1 | 6 | 7.5 | 7.5 |
| Petioles @ midrib (mm) | | | | | | |
| Mean Depth of Outer | 12.9 | 6.5 | 6.5 | 3.9 | 4.6 | 6.6 |
| Petioles @ midrib (mm) | | | | | | |
| Mean Number of 7 inch | 14.9 | 1.1 | 0 | 7 | 7.6 | 11.7 |
| Straws per Plant | | | | | | |
| Mean Straw Yield per | 0.309 | 0.012 | 0 | 0.044 | 0.061 | 0.084 |
| Plant (kg) | | | | | | |
| Mean Weight per Straw | 20.7 | 10.5 | 0 | 6.3 | 8 | 7.2 |
| (g) | | | | | | |
| Number 7″ Straws per | 865,392 | 63,888 | 0 | 406,560 | 441,408 | 679,536 |
| Acre | | | | | | |
| Straw Yield per Acre (kg) | 17,947 | 668 | 0 | 2,556 | 3,543 | 4,879 |
| Mean Vacuum (in/Hg) | 27.4 | 10.7 | 11.2 | 11.5 | 12.9 | 14.0 |
| Mean Wall Thickness at | 3.29 | 1.82 | 2.07 | 1.26 | 1.01 | 1.42 |
| Sides of Petiole (mm) | | | | | | |
| Mean Wall Thickness at | 1.1 | 0.75 | 0.55 | 0.39 | 0.54 | 0.37 |
| Inside of Petiole Cup | | | | | | |
| (mm) | | | | | | |
| Mean Pressure Required | 3186 | 1554 | 1630 | 1055 | 990 | 698 |
| to Rupture Side Wall (g) | | | | | | |
| Mean Pressure Required | 853 | 417 | 444 | 414 | 292 | 145 |
| to Rupture Wall @ Inside | | | | | | |
| Of Petiole Cup (g) | | | | | | |
| Mean Percentage Dry | 6.5% | 8.0% | 7.7% | 8.5% | 8.2% | 9.2% |
| Weight | | | | | | |

Table 8 shows a comparison between ADS-9 of the present invention, Afina, a hollow-stem leaf celery (Apium graveolens L. var secalinum) and numerous hollow-stem celeriacs (Apium graveolens L. var rapaceum) including commercial and representatives from the United States germplasm collection. All comparisons were generated from a trial harvested May 29, 2008 in Oxnard, Calif. The trial was transplanted Feb. 27, 2008 at a population of 58,080 plants per acre. Production was during a period when conditions were fairly normal and free from most stresses. This data shows that there may have been marginal bolting pressure, but pressure was light and the presence of seed stems as an indication that the varieties were particularly sensitive. Harvest occurred May 29, 2008 at 92 days maturity.

TABLE 8

| | | Blanco de | Monarch | | | |
|---|---|---|---|---|---|---|
| | ADS-9 | Veneto | Apium | Afina | PI 193454 | PI 179171 |
| | Apium | Apium | graveolens | Apium | Apium | Apium |
| | graveolens | graveolens L. var | L. var | graveolens L. var | graveolens L. var | graveolens L. var |
| | L. var dulce | rapaceum | rapaceum | secalinum | rapaceum | rapaceum |
| Mean Plant Height (cm) | 58.7 | 49.3 | 45.9 | 55.6 | 53.9 | 67.9 |
| Mean Plant Width (cm) | 42.3 | 36.1 | 27.7 | 41.6 | 44.4 | 45.0 |
| Mean Whole Plant weight | 0.3655 | 0.3295 | 0.1935 | 0.198 | 0.295 | 0.249 |
| (kg) | | | | | | |
| Mean Trimmed Plant | 17.9 | 0.0 | 0.0 | 103.8 | 0.0 | 25.8 |
| Weight (kg) | | | | | | |
| Mean Number of Suckers | 0.0 | 56.8 | 61.9 | 0.0 | 82.0 | 66.0 |
| Mean Root Diameter (cm) | 0.0 | 64.2 | 76.1 | 0.0 | 96.0 | 80.0 |
| Mean Root Depth (cm) | 0.0 | 0.114 | 0.115 | 0.0 | 0.258 | 0.213 |
| Mean Root weight (kg) | 36.9 | 20.0 | 17.8 | 24.8 | 27.8 | 37.6 |
| Mean Joint Length (cm) | 11.2 | 9.7 | 12.1 | 7.4 | 12.6 | 11.9 |
| Mean Number of Outer | 7.8 | 9.8 | 6.5 | 9.2 | 7.1 | 5.0 |
| Petioles (>40 cm) | | | | | | |
| Mean Number of Inner | 1.5 | 0.0 | 0.0 | 0.0 | 0.5 | 1.2 |
| Petioles (<40 cm) | | | | | | |
| Mean Seed Stem Length | 11.4 | 7.3 | 7.1 | 6 | 7.5 | 7.5 |
| (cm) | | | | | | |
| Mean Width of Outer | 10.1 | 6.5 | 6.5 | 3.9 | 4.6 | 6.6 |
| Petioles @ midrib (mm) | | | | | | |
| Mean Number of 7 inch | 24 | 1.1 | 0 | 7 | 7.6 | 11.7 |
| Straws per Plant | | | | | | |
| Mean Straw Yield per | 0.251 | 0.012 | 0 | 0.044 | 0.061 | 0.084 |
| Plant (kg) | | | | | | |
| Mean Weight per Straw | 10.5 | 10.5 | 0 | 6.3 | 8 | 7.2 |
| (g) | | | | | | |
| Number 7″ Straws per | 1,393,920 | 63,888 | 0 | 406,560 | 441,408 | 679,536 |
| Acre | | | | | | |
| Straw Yield per Acre (kg) | 14,578 | 668 | 0 | 2,556 | 3,543 | 4,879 |
| Mean Vacuum (in/Hg) | 29.6 | 10.7 | 11.2 | 11.5 | 12.9 | 14.0 |
| Mean Wall Thickness at | 2.05 | 1.82 | 2.07 | 1.26 | 1.01 | 1.42 |
| Sides of Petiole (mm) | | | | | | |
| Mean Wall Thickness at | 0.67 | 0.75 | 0.55 | 0.39 | 0.54 | 0.37 |
| Inside of Petiole Cup | | | | | | |
| (mm) | | | | | | |
| Mean Pressure Required | 2229 | 1554 | 1630 | 1055 | 990 | 698 |
| to Rupture Side Wall (g) | | | | | | |
| Mean Pressure Required | 1208 | 417 | 444 | 414 | 292 | 145 |
| to Rupture Wall @ Inside | | | | | | |
| Of Petiole Cup (g) | | | | | | |
| Mean Percentage Dry | 6.7% | 8.0% | 7.7% | 8.5% | 8.2% | 9.2% |
| Weight | | | | | | |

Table 9 shows a comparison between ADS-19 of the present invention and numerous celeriacs (Apium graveolens L. var rapaceum) including commercial and representatives from the United States germplasm collection. All comparisons were generated from a trial harvested May 29, 2008 in Oxnard, Calif. The trial was transplanted Feb. 27, 2008 at a population of 58,080 plants per acre. Production was during a period when conditions were fairly normal and free from most stresses. This data shows that there may have been marginal bolting pressure, but pressure was light and the presence of seed stems an indication that the varieties were particularly sensitive. Harvest occurred May 29, 2008 at 92 days maturity.

TABLE 9

| | ADS-19 | PI 261810* | PI 164944 | PI 169001 | PI 176417 | PI 178834 |
|---|---|---|---|---|---|---|
| | Apium | Apium | Apium | Apium | Apium | Apium |
| | graveolens | graveolens L. var | graveolens L. var | graveolens L. var | graveolens L. var | graveolens L. var |
| | L. var dulce | rapaceum | rapaceum | rapaceum | rapaceum | rapaceum |
| Mean Plant Height (cm) | 91.4 | 49.5 | 81.0 | 61.2 | 63.3 | 81.0 |
| Mean Plant Width (cm) | 34.8 | 42.6 | 49.5 | 52.7 | 52.8 | 52.2 |
| Mean Whole Plant weight | 0.967 | 0.426 | 0.647 | 0.569 | 0.474 | 0.724 |
| (kg) | | | | | | |
| Mean Trimmed Plant | 0.901 | 0.308 | 0.266 | 0.398 | 0.274 | 0.43 |
| Weight (kg) | | | | | | |
| Mean Number of Suckers | 0.0 | 3.5 | 49.6 | 18.3 | 35.0 | 40.0 |
| Mean Root Diameter (cm) | 0.0 | 90.0 | 70.0 | 70.0 | 48.3 | 47.2 |
| Mean Root Depth (cm) | 0.0 | 100.0 | 80.0 | 72.0 | 74.0 | 78.0 |
| Mean Root weight (kg) | 0.0 | 0.375 | 0.235 | 0.212 | 0.073 | 0.23 |
| Mean Joint Length (cm) | 44.6 | 24.0 | 40.6 | 31.0 | 36.1 | 47.2 |
| Mean Number of Outer | 10.0 | 6.1 | 12.8 | 16.5 | 13.6 | 14.9 |
| Petioles (>40 cm) | | | | | | |
| Mean Number of Inner | 6.6 | 17.3 | 8.1 | 6.4 | 5.0 | 4.8 |
| Petioles (<40 cm) | | | | | | |
| Mean Seed Stem Length | 0.0 | 0.0 | 47.7 | 4.4 | 4.0 | 1.9 |
| (cm) | | | | | | |
| Mean Width of Outer | 18.1 | 7.7 | 7.6 | 7.7 | 6.7 | 7.3 |
| Petioles @ midrib (mm) | | | | | | |
| Mean Depth of Outer | 11.9 | 3.5 | 5.6 | 5.5 | 4.7 | 5.8 |
| Petioles @ midrib (mm) | | | | | | |
| Mean Number of 7 inch | 12.4 | 6.5 | 16.3 | 12.9 | 13.4 | 20.1 |
| Straws per Plant | | | | | | |
| Mean Straw Yield per | 0.320 | 0.054 | 0.103 | 0.103 | 0.094 | 0.115 |
| Plant (kg) | | | | | | |
| Mean Weight per Straw | 25.8 | 8.3 | 6.3 | 8 | 7 | 8.2 |
| (g) | | | | | | |
| Number 7″ Straws per | 720,192 | 377,520 | 946,704 | 749,232 | 778,272 | 1,167,408 |
| Acre | | | | | | |
| Straw Yield per Acre (kg) | 18,586 | 3,136 | 5,982 | 5,982 | 5,460 | 6,679 |
| Mean Vacuum (in/Hg) | 25.3 | 12.7 | 18.1 | 15.3 | 16.9 | 10.6 |
| Mean Wall Thickness at | 4.18 | 1.21 | 1.47 | 1.56 | 1.5 | 1.6 |
| Sides of Petiole (mm) | | | | | | |
| Mean Wall Thickness at | 1.44 | 0.41 | 0.44 | 0.66 | 0.51 | 0.54 |
| Inside of Petiole Cup | | | | | | |
| (mm) | | | | | | |
| Mean Pressure Required | 2105 | 657 | 1205 | 1062 | 1370 | 1296 |
| to Rupture Side Wall (g) | | | | | | |
| Mean Pressure Required | 661 | 123 | 375 | 486 | 467 | 415 |
| to Rupture Wall @ Inside | | | | | | |
| Of Petiole Cup (g) | | | | | | |
| Mean Percentage Dry | 6.2% | 9.2% | 9.4% | 9.0% | 9.7% | 8.7% |
| Weight | | | | | | |

Table 10 shows a comparison between ADS-15 of the present invention and numerous celeriacs (Apium graveolens L. var rapaceum) including commercial and representatives from the United States germplasm collection. All comparisons were generated from a trial harvested May 29, 2008 in Oxnard, Calif. The trial was transplanted Feb. 27, 2008 at a population of 58,080 plants per acre. Production was during a period when conditions were fairly normal and free from most stresses. This data shows that there may have been marginal bolting pressure, but pressure was light and the presence of seed stems an indication that the varieties were particularly sensitive. Harvest occurred May 29, 2008 at 92 days maturity.

TABLE 10

| | ADS-15 | PI 261810* | PI 164944 | PI 169001 | PI 176417 | PI 178834 |
|---|---|---|---|---|---|---|
| | Apium | Apium | Apium | Apium | Apium | Apium |
| | graveolens | graveolens L. var | graveolens L. var | graveolens L. var | graveolens L. var | graveolens L. var |
| | L. var dulce | rapaceum | rapaceum | rapaceum | rapaceum | rapaceum |
| Mean Plant Height (cm) | 98.7 | 49.5 | 81.0 | 61.2 | 63.3 | 81.0 |
| Mean Plant Width (cm) | 33.7 | 42.6 | 49.5 | 52.7 | 52.8 | 52.2 |
| Mean Whole Plant weight | 0.851 | 0.426 | 0.647 | 0.569 | 0.474 | 0.724 |
| (kg) | | | | | | |
| Mean Trimmed Plant | 0.673 | 0.308 | 0.266 | 0.398 | 0.274 | 0.43 |
| Weight (kg) | | | | | | |
| Mean Number of | 0.0 | 3.5 | 49.6 | 18.3 | 35.0 | 40.0 |
| Suckers | | | | | | |
| Mean Root Diameter | 0.0 | 90.0 | 70.0 | 70.0 | 48.3 | 47.2 |
| (cm) | | | | | | |
| Mean Root Depth (cm) | 0.0 | 100.0 | 80.0 | 72.0 | 74.0 | 78.0 |
| Mean Root weight (kg) | 0.0 | 0.375 | 0.235 | 0.212 | 0.073 | 0.23 |
| Mean Joint Length (cm) | 49.4 | 24.0 | 40.6 | 31.0 | 36.1 | 47.2 |
| Mean Number of Outer | 9.6 | 6.1 | 12.8 | 16.5 | 13.6 | 14.9 |
| Petioles (>40 cm) | | | | | | |
| Mean Number of Inner | 5.6 | 17.3 | 8.1 | 6.4 | 5.0 | 4.8 |
| Petioles (<40 cm) | | | | | | |
| Mean Seed Stem Length | 0.0 | 0.0 | 47.7 | 4.4 | 4.0 | 1.9 |
| (cm) | | | | | | |
| Mean Width of Outer | 16.9 | 7.7 | 7.6 | 7.7 | 6.7 | 7.3 |
| Petioles @ midrib (mm) | | | | | | |
| Depth of Outer Petioles | 12.9 | 3.5 | 5.6 | 5.5 | 4.7 | 5.8 |
| @ midrib (mm) | | | | | | |
| Mean Number of 7 inch | 14.9 | 6.5 | 16.3 | 12.9 | 13.4 | 20.1 |
| Straws per Plant | | | | | | |
| Mean Straw Yield per | 0.309 | 0.054 | 0.103 | 0.103 | 0.094 | 0.115 |
| Plant (kg) | | | | | | |
| Mean Weight per Straw | 20.7 | 8.3 | 6.3 | 8 | 7 | 8.2 |
| (g) | | | | | | |
| Number 7″ Straws per | 865,392 | 377,520 | 946,704 | 749,232 | 778,272 | 1,167,408 |
| Acre | | | | | | |
| Straw Yield per Acre (kg) | 17,947 | 3,136 | 5,982 | 5,982 | 5,460 | 6,679 |
| Mean Vacuum (in/Hg) | 27.4 | 12.7 | 18.1 | 15.3 | 16.9 | 10.6 |
| Mean Wall Thickness at | 3.29 | 1.21 | 1.47 | 1.56 | 1.5 | 1.6 |
| Sides of Petiole (mm) | | | | | | |
| Mean Wall Thickness at | 1.1 | 0.41 | 0.44 | 0.66 | 0.51 | 0.54 |
| Inside of Petiole Cup | | | | | | |
| (mm) | | | | | | |
| Mean Pressure Required | 3186 | 657 | 1205 | 1062 | 1370 | 1296 |
| to Rupture Side Wall (g) | | | | | | |
| Mean Pressure Required | 853 | 123 | 375 | 486 | 467 | 415 |
| to Rupture Wall @ Inside | | | | | | |
| Of Petiole Cup (g) | | | | | | |
| Mean Percentage Dry | 6.5% | 9.2% | 9.4% | 9.0% | 9.7% | 8.7% |
| Weight | | | | | | |

Table 11 shows a comparison between ADS-9 of the present invention and numerous celeriacs (Apium graveolens L. var rapaceum) including commercial and representatives from the United States germplasm collection. All comparisons were generated from a trial harvested May 29, 2008 in Oxnard, Calif. The trial was transplanted Feb. 27, 2008 at a population of 58,080 plants per acre. Production was during a period when conditions were fairly normal and free from most stresses. This data shows that there may have been marginal bolting pressure, but pressure was light and the presence of seed stems an indication that the varieties were particularly sensitive. Harvest occurred May 29, 2008 at 92 days maturity.

TABLE 11

| | | PI 261810* | | | | |
|---|---|---|---|---|---|---|
| | ADS-9 | Apium | PI 164944 | PI 169001 | PI 176417 | PI 178834 |
| | Apium | graveolens | Apium | Apium | Apium | Apium |
| | graveolens | L. var | graveolens L. var | graveolens L. var | graveolens L. var | graveolens L. var |
| | L. var dulce | rapaceum | rapaceum | rapaceum | rapaceum | rapaceum |
| Mean Plant Height (cm) | 58.7 | 49.5 | 81.0 | 61.2 | 63.3 | 81.0 |
| Mean Plant Width (cm) | 42.3 | 42.6 | 49.5 | 52.7 | 52.8 | 52.2 |
| Mean Whole Plant weight | 0.417 | 0.426 | 0.647 | 0.569 | 0.474 | 0.724 |
| (kg) | | | | | | |
| Mean Trimmed Plant | 0.3655 | 0.308 | 0.266 | 0.398 | 0.274 | 0.43 |
| Weight (kg) | | | | | | |
| Mean Number of | 17.9 | 3.5 | 49.6 | 18.3 | 35.0 | 40.0 |
| Suckers | | | | | | |
| Mean Root Diameter | 0.0 | 90.0 | 70.0 | 70.0 | 48.3 | 47.2 |
| (cm) | | | | | | |
| Mean Root Depth (cm) | 0.0 | 100.0 | 80.0 | 72.0 | 74.0 | 78.0 |
| Mean Root weight (kg) | 0.0 | 0.375 | 0.235 | 0.212 | 0.073 | 0.23 |
| Mean Joint Length (cm) | 36.9 | 24.0 | 40.6 | 31.0 | 36.1 | 47.2 |
| Mean Number of Outer | 11.2 | 6.1 | 12.8 | 16.5 | 13.6 | 14.9 |
| Petioles (>40 cm) | | | | | | |
| Mean Number of Inner | 7.8 | 17.3 | 8.1 | 6.4 | 5.0 | 4.8 |
| Petioles (<40 cm) | | | | | | |
| Mean Seed Stem Length | 1.5 | 0.0 | 47.7 | 4.4 | 4.0 | 1.9 |
| (cm) | | | | | | |
| Mean Width of Outer | 11.4 | 7.7 | 7.6 | 7.7 | 6.7 | 7.3 |
| Petioles @ midrib (mm) | | | | | | |
| Depth of Outer Petioles | 10.1 | 3.5 | 5.6 | 5.5 | 4.7 | 5.8 |
| @ midrib (mm) | | | | | | |
| Mean Number of 7 inch | 24 | 6.5 | 16.3 | 12.9 | 13.4 | 20.1 |
| Straws per Plant | | | | | | |
| Mean Straw Yield per | 0.251 | 0.054 | 0.103 | 0.103 | 0.094 | 0.115 |
| Plant (kg) | | | | | | |
| Mean Weight per Straw | 10.5 | 8.3 | 6.3 | 8 | 7 | 8.2 |
| (g) | | | | | | |
| Number 7″ Straws per | 1,393,920 | 377,520 | 946,704 | 749,232 | 778,272 | 1,167,408 |
| Acre | | | | | | |
| Straw Yield per Acre (kg) | 14,578 | 3,136 | 5,982 | 5,982 | 5,460 | 6,679 |
| Mean Vacuum (in/Hg) | 29.6 | 12.7 | 18.1 | 15.3 | 16.9 | 10.6 |
| Mean Wall Thickness at | 2.05 | 1.21 | 1.47 | 1.56 | 1.5 | 1.6 |
| Sides of Petiole (mm) | | | | | | |
| Mean Wall Thickness at | 0.67 | 0.41 | 0.44 | 0.66 | 0.51 | 0.54 |
| Inside of Petiole Cup | | | | | | |
| (mm) | | | | | | |
| Mean Pressure Required | 2229 | 657 | 1205 | 1062 | 1370 | 1296 |
| to Rupture Side Wall (g) | | | | | | |
| Mean Pressure Required | 1208 | 123 | 375 | 486 | 467 | 415 |
| to Rupture Wall @ Inside | | | | | | |
| Of Petiole Cup (g) | | | | | | |
| Mean Percentage Dry | 6.7% | 9.2% | 9.4% | 9.0% | 9.7% | 8.7% |
| Weight | | | | | | |

As shown in Tables 4, 5, 8 and 11, ADS-9 unexpectedly demonstrates a very slight to moderate sucker count. A unexpected significant difference between ADS-9, ADS-15, ADS-19 and Afina with the root celery lines (Apium graveolens L. var rapaceum) is the absence of a swollen root (tuber) as shown in Tables 7, 8, 9, 10 and 11. While the maturity was not sufficient to allow for maximum swelling/yield there was obvious and measureable swelling. Measurements were taken for width, depth and weight. No swelling had occurred in the stem celery (Apium graveolens L. var dulce) or leaf celery leaf celery (Apium graveolens L. var secalinum). While bolting pressure was very light PI 164944 was obviously very susceptible to bolting. Differences between the hollow petiole Apium graveolens L. var dulce types when compared with all other classes became especially pronounced when the petiole width and thickness was measured at the mid-rib. ADS-15 was unexpectedly 125% wider than non Apium graveolens L. var rapaceum types and ADS-19 was 7% wider than ADS-15. Both were significantly wider than ADS-9. The total number of straws or 7-inch segments varied among all of the lines but when all characteristics, including width thickness, vacuum, rupture pressure and wall thickness were considered only ADS-9, ADS-15 and ADS-19 made reasonably usable straws. When comparing wall thickness, vacuum and rupture pressure ADS-9, ADS-15 and ADS-19 were unexpectedly much more durable than all other lines tested.

Table 12 shows a comparison between ADS-19, ADS-9 and ADS-15 of the present invention, two root celeries, Monarch and Blanco de Veneto (Apium graveolens L. var rapaceum) and Afina a leaf celery (Apium graveolens L. var secalinum) in a trial grown in Salinas, Calif. The trial was transplanted Apr. 22, 2007 at a population of 63,000 plants per acre. Production was under normal conditions with no stresses. Harvest occurred Jul. 23, 2007 at 92 days maturity.

TABLE 12

| | | | | | | Blanco De |
|---|---|---|---|---|---|---|
| | | | | Afina | Monarch | Veneto |
| | ADS-15 | ADS-9 | ADS-19 | Apium | Apium | Apium |
| | Apium | Apium | Apium | graveolens | graveolens | graveolens |
| | graveolens | graveolens | graveolens | L. var | L. var | L. var |
| | L. var dulce | L. var dulce | L. var dulce | secalinum | rapaceum | rapaceum |
| Mean Width | 17.9 | 12.3 | 21.1 | 5.5 | 6.5 | 6 |
| of Outer | | | | | | |
| Petioles @ | | | | | | |
| midrib (mm) | | | | | | |
| Mean Depth | 14 | 9.9 | 16.4 | 5 | 6 | 6 |
| of Outer | | | | | | |
| Petioles @ | | | | | | |
| midrib (mm) | | | | | | |
| Mean | 15.9 | 17.4 | 12.4 | 8.4 | 9.1 | 10.2 |
| Vacuum | | | | | | |
| (in/Hg) | | | | | | |
| Mean Wall | 3.5 | 3.0 | 4.8 | 1.2 | 1.3 | 1.3 |
| Thickness at | | | | | | |
| Sides of | | | | | | |
| Petiole (mm) | | | | | | |
| Mean Wall | 1.5 | 1.0 | 1.5 | 0.7 | 0.7 | 0.7 |
| Thickness at | | | | | | |
| Inside of | | | | | | |
| Petiole Cup | | | | | | |
| (mm) | | | | | | |
| Mean | 1921 | 2050 | 2166 | 1521 | 975 | 873 |
| Pressure | | | | | | |
| Required to | | | | | | |
| Rupture Side | | | | | | |
| Wall (grams) | | | | | | |
| Mean | 454 | 700 | 434 | 201 | 195 | 150 |
| Pressure | | | | | | |
| Required to | | | | | | |
| Rupture Wall | | | | | | |
| @ Inside of | | | | | | |
| Petiole Cup | | | | | | |
| (grams) | | | | | | |
| Mean Percent | 7.6% | 8.6% | 7.4% | 9.1% | 9.1% | 10.2% |
| dry weight | | | | | | |

As can be seen in Table 12 the petiole widths of ADS-19, ADS-15 and ADS-9 (Apium graveolens L. var dulce) are unexpectedly significantly greater than the petiole widths of the leaf celery (Apium graveolens L. var secalinum) and root celery (Apium graveolens L. var rapaceum) varieties. The petiole of ADS-9 is approximately 100% larger while the petioles of ADS-15 and ADS-19 are at least 175% wider than Afina, Monarch and Blanco de Veneto (Apium graveolens L. var secalinum and Apium graveolens L. var rapaceum). Similarly the depth of the outer petioles at the midrib was unexpectedly significantly larger than the celeriac and leaf celery varieties with ADS-9 (65% deeper) and ADS-15 and ADS-19 (at least 133%) deeper than Afina, Monarch and Blanco de Veneto (Apium graveolens L. var secalinum and Apium graveolens L. var rapaceum). In combination with thicker side walls, 130% to 269% thicker, a greater capacity to withstand a vacuum, 22% to 71% more resilient and greater pressure required to rupture the walls, the ADS-19, ADS-15 and ADS-9 varieties are more suited for use as a straw. When ADS-15 and ADS-19 are compared directly, ADS-19 is found to be more appropriate for use as a consumed or edible product to be stuffed with edible material, while ADS-15 is more appropriate for use as a straw. ADS-19 has petioles that are 18% wider and 17% deeper than ADS-15 making a larger hollow tube more suitable for stuffing. The petioles of ADS-19 are also 71% thicker and have a lower percentage dry weight than ADS-15. This unexpected lower percentage dry weight correlates with ADS-19 being juicier, less fibrous and much more edible. ADS-15 on the other hand is more suited to a straw with slightly smaller diameter.

Table 13 shows a comparison between ADS-15 and ADS-19 of the present invention in a trial grown in Oxnard, Calif. for the purpose of evaluating the varieties for tolerance to Fusarium oxysporum f. sp. apii race 2. The trial was transplanted Aug. 13, 2008 at a population of 50,000 plants per acre. This trial was sown in a research plot that has been specially developed with elevated fusarium levels.

TABLE 13

| | ADS-15 | ADS-19 |
|---|---|---|
| | Apium | Apium |
| | graveolens | graveolens |
| | L. var dulce | L. var dulce |
| Mean Plant Height (cm) | 107.6 | 102.6 |
| Mean Whole Plant weight (kg) | 0.956 | 0.764 |
| Mean Trim Plant weight (kg) | 0.729 | 0.589 |
| Mean Number of Suckers | 0 | 0 |
| Mean Joint Length (cm) | 47.9 | 50.2 |
| Mean Number of Outer Petioles | 11.3 | 8.9 |
| >40 cm | | |
| Mean Number of Inner Petioles <40 cm | 1.8 | 2.1 |
| Mean Width of Outer Petioles @ | 15.1 | 15.8 |
| midrib (mm) | | |
| Mean Depth of Outer Petioles @ | 13.1 | 13.0 |
| midrib (mm) | | |
| Mean Wall Thickness at Sides of | 2.9 | 3.4 |
| Petiole (mm) | | |
| Mean Number of 7 inch Straws per | 17.4 | 14.5 |
| Plant | | |
| Mean Straw Yield per Plant (kg) | 0.319 | 0.338 |
| Mean Weight per Straw (g) | 0.018 | 0.023 |
| Number of 7 inch Straws per Acre | 870,000 | 725,000 |
| Straw Yield per Acre (kg) | 15,950 | 16,900 |
| Mean General Fusarium Rating | 4 | 3.5 |
| (0 = death to 5 = resistant) | | |
| Mean Fusarium Injury in the Root | 4 | 3.0 |
| (0 = death to 5 = resistant) | | |

As can be seen in Table 13, a comparison of ADS-15 and ADS-19 as they performed under conditions with no Fusarium (Tables 2 and 3) to these conditions where Fusarium was severe indicate that there was little impact on the size, yield, length and number of the straws generated. While there was some Fusarium present as evidenced by the general Fusarium and root Fusarium ratings, however the economic impact was not significant. The data indicate that celery cultivar ADS-15 has better tolerance to Fusarium than celery cultivar ADS-19.

### Example 3

### Preparation of the Hollow Celery Stick

In the preparation of a product for the marketplace, raw hollow celery sticks of the present invention are harvested by hand or machine in the field similar to standard stem celery and placed in bins, totes or cartons and cooled. Cooling will usually be performed by utilizing hydro-vac cooling, hydro cooling or forced aircooling methods typical of most raw vegetables, but other methods may be used.

The initial processing steps may include cleaning steps often used for raw vegetables to assure cleanliness and food safety.

Once the hollow celery of the present invention is cooled, the cool process will be maintained within the range of 33° F. to 40° F. unless the specific product or process requires a break in that chain.

In the present invention, a whole celery stalk is trimmed to remove the butt and foliage. The celery may either be cut by hand or mechanical means such as a saw or knife. It may also be cut with a water knife or similar type advancements in cutting technology.

The celery of the present invention is a novel and natural straw that can be utilized for the consumption of beverages and is the result of cutting the petioles of hollow celery in a manner that maintains the integrity of the celery. Maintenance of the integrity of the stem is especially critical because holes or cracks in the product/straw will result in an unusable product.

The water knife/water jet cutter is a special technology which has been adopted specially for cutting hollow celery of the present invention. Due to the hollow nature of the product, conventional knives and saws have a greater opportunity to collapse the straw at the point of impact, thus causing the straw to rupture, split or crack. Once a straw is damaged it is difficult to consume a beverage through it.

However, the water knife cuts by a very high pressure stream of water and air (over 37,000 psi) being passed through a very small orifice or nozzle (approximately, but not limited to, 0.0007 cm in diameter). The hollow celery passing through this high pressure stream of air and water is cut with no risk of straw collapse, because no physical pressure is applied to the hollow celery. See, for example, U.S. Pat. Nos. 3,974,725, 4,601,156, 4,753,808, 6,308,600, 4,751,094, and 5,916,354.

Water is first run through a pre-chiller which drops the temperature to between 34° and 36° F. The water is then run into an intensifier where it is run through a filtration system to remove impurities. The intensifier then compresses water and air independently. A stream of water is then injected into the air such that the air acts as the carrier and the water as the abrasive. This mixture passes through a set of cutting nozzles (>37,000 psi) that have an orifice between 0.0003 cm and 0.0010 cm in diameter.

Several water jet nozzles are placed in series at intervals that match the length of the celery straws that are desired. The whole hollow celery stalk is then passed through these nozzles or knives on a conveyor. As the celery stalk passes though the pressurized water emanating from each nozzle the celery stalk is cut into a celery stick. The length of the celery stick will depend on the specific product or the requirements of the consumer.

Additional cleaning steps typical of raw, semi-processed and processed vegetables may be utilized to assure cleanliness and food safety. These steps may include chlorine or other cleansing/sterilizers in rinse water applied via a drenching or water bath system. Typical solutions include chlorine and water at a concentration of approximately 750 ppm or cleansing/sterilizers at their suggested use rates. Other technologies may be utilized as appropriate or acceptable.

All products should be handled from this step forward in an aseptic environment following general HACCP procedures in order to ensure food safety.

The cut and cleaned hollow celery stick of the present invention is sorted or graded by size and quality based on standards established for the specific products, uses or customer requirements. The celery is then run through a metal detector following processing or prior to or following packaging to ensure that no metal has contaminated the product.

Hollow celery sticks of the present invention are packaged and sealed in various container types according to the customer's requirements. The cool process remains unbroken for this product, as it is sold as a perishable product.

Hollow celery sticks may be handled in a slightly dehydrated (wilted or limber) state and then rehydrated by the consumer by placing in a container with clean water for several minutes. The consumer may trim the ends with a knife to improve freshness, appearance, or adjust the length.

### Example 4

### Injection of Food into the Hollow Celery Stick

Prior to delivery of the hollow celery sticks of the present invention to the food injection site, the hollow celery stick will need to undergo a sanitation process. The celery stick may be sanitized by any number of methods which include but are not limited to, the following: ascorbic acid and peroxyacetic acid also known as TSUNAMI, sodium hypochlorite (chlorine), bromine products (sodium hypobromine), chlorine dioxide, ozone based systems, hydrogen peroxide products, trisodium phosphate, quaternary ammonium products, ultraviolet light systems, solar radiation, nuclear radiation, irradiation, steam, ultra heat treatments, ultra cold treatments and high pressure pasteurization. See, for example, U.S. Pat. Nos. 7,220,381, 5,945,146 and 4,753,808 and US Publication No. 2004/0191382, Zagory, D. 1999. Liao, C., Cooke, P. H. 2001. Response to Trisodium Phosphate Treatment of Salmonella Chester Attached to Fresh-Cut Green Pepper Slices. Canadian J. Micro. 47:25-32; Jongen, W., ed. 2005. Improving the Safety of Fresh Fruit and Vegetables. C.H.I.P.S., Weimar, Tex.

Another method that could be used to sanitize and wash the celery sticks of the present invention would be the use of cool or warm air that is blown through the hollow celery stick, the hollow celery is then placed in a centrifuge or shaker to remove as much moisture as possible, the straws are then allowed to naturally drain by standing on end. The hollow celery sticks are then placed in a vacuum to remove all additional moisture and finally placed in a desiccator.

Once the celery of the present invention has been sanitized, raw or cooked hollow celery stick may be stuffed with an injection system specifically modified to match the diameter of the hollow celery stick and the consistency of the food product being injected.

Possible methods of delivering the hollow celery sticks of the present invention to the food injection location for injecting various food products into the hollow celery sticks include but are not limited to mechanical means including hydraulic, pneumatic and electrical. The equipment that is used for these methods includes but is not limited to belt conveyors, flat, flighted or grooved, made of vinyl or rubber. Shakers or vibrating machines for up and down and/or side to side motions or fast back and forth motions could also be used. Step-motion orientators that create a back and forth, side to side or up and down motion or elevator orientators that create vertical or any degree of incline or decline could be used as well as transfer slides that create any degree of incline or decline. Finally, transfer belts, including flat, flighted and grooved made of plastic or rubber could be used to deliver the celery to the food product injection location.

Delivery of the hollow celery sticks and the food product to the injection location may also take place manually and include equipment such as containers, tables, transfer jigs, placement jigs, and semi-automatic feeders.

Once the hollow celery sticks and the food product have been delivered to the injection site the method of injection may include but is not limited to: hydraulic, pneumatic, electrical or water injection. The equipment that may be used in this process includes but is not limited to injection needles and injection tubes. The force required to inject the food product into the hollow celery stick could be created through force air or vacuum pressure based on either positive or negative pressure. The force required to inject the food product into the hollow celery sticks could also be created through forced or vacuum water pressure under either positive or negative pressure.

The injection of the food product into the hollow celery sticks could also be done manually. The equipment that may be necessary for this process includes but is not limited to injections needles, injection tubes, plastics or rubber basters, pastry bags or frosting bags, frosting tips, and semi-automatic injectors.

Depending on the particular food product being injected, another cooling procedure may be required to re-establish an appropriate temperature. This cooling procedure, if required, may take place prior to or just following packaging in customer specified packaging. The cold process must be maintained throughout shipment and delivery to the customer.

Each type of cooked, stuffed product may have an entirely different treatment or preparation process.

Once the celery product of the present invention is sanitized and injected with the appropriate food product, it is packaged according to length and may be packaged in any number of methods according to the specifications of the customer. The product of the present invention may be packaged in numerous types of packages including but not limited to rigid plastic, flexible film, solid fiber, poly sleeves, plastic sleeves, poly bags, plastic bags, natural decomposable bags, natural decomposable sleeves, packages that may be opened and sealed, rigid containers like clam shells, packages with different permeability properties, packages with built-in vents, packages with specialized pores or any combination thereof, or any combination thereof. Variations in the packaging may include different gas exchange rates which may occur due to different permeability or transmission properties of the package materials themselves or due to vents or specialized pores built into the packaging. See for example, U.S. Pat. Nos. 4,753,808, and 4,586,313.

### Example 5

### An Example of the Preparation and Injection Process of the Hollow Celery Stick

The edible, hollow celery of the present invention is field harvested in bulk bins (101.6 cm×121.9 cm×121.98 cm) and delivered to a processing plant. The bins are placed in a Hydro-Vac tube where sanitized spray water is added and then a vacuum is drawn to bring the product temperature to approximately 34° F.

The celery of the present invention is placed on a conveyor belt, oriented so that it can pass through a water knife to cut off the butt or base of the plant and the top or leaves. The water knives are set to obtain the length desired for the straws or stuffed product. The celery butt and tops fall off the belt and the cut sections are conveyed through a chilled, disinfecting water shower.

The hollow celery stick of the present invention will then need to undergo a sanitation process. The celery stick may be sanitized by any number of methods which include but are not limited to, the following: ascorbic acid and peroxyacetic acid also known as TSUNAMI, sodium hypochlorite (chlorine), bromine products (sodium hypobromine), chlorine dioxide, ozone based systems, hydrogen peroxide products, trisodium phosphate, quaternary ammonium products, ultraviolet light systems, solar radiation, nuclear radiation, irradiation, steam, ultra heat treatments, ultra cold treatments and high pressure pasteurization. See, for example, U.S. Pat. Nos. 7,220,381, 5,945,146 and 4,753,808 and US Publication No. 2004/0191382, Zagory, D. 1999. Liao, C., Cooke, P. H. 2001. Response to Trisodium Phosphate Treatment of Salmonella Chester Attached to Fresh-Cut Green Pepper Slices. Canadian J. Micro. 47:25-32; Jongen, W., ed. 2005. Improving the Safety of Fresh Fruit and Vegetables. C.H.I.P.S., Weimar, Tex.

Another method that could be used to sanitize and wash the celery sticks would be the use of cool or warm air that is blown through the hollow celery stick, the hollow celery is then placed in a centrifuge or shaker to remove as much moisture as possible, the straws are then allowed to naturally drain by standing on end. The hollow celery sticks are then placed in a vacuum to remove all additional moisture and finally placed in a desiccator.

Additional cleaning steps typical of raw, semi-processed and processed vegetables may be utilized to assure cleanliness and food safety. These steps may include chlorine or other cleansing/sterilizers in rinse water applied via a drenching or water bath system. Typical solutions include chlorine and water at a concentration of approximately 750 ppm or cleansing/sterilizers at their suggested use rates. Other technologies may be utilized as appropriate or acceptable.

All products should be handled from this step forward in an aseptic environment following general HACCP procedures in order to ensure food safety.

These sections then pass through an air shower to remove excessive moisture, and the conveyer belt continues to carry the sections to a sorting station where the product is graded manually to meet the standards for the product and customer.

If the product is a straw, the straws are then carried via a conveyor to packing stations where they are manually packed in the appropriate packaging.

If packed in poly sealed bags the packages are run through a sealer where they are date stamped with a “use by” date specific to the specific product. They continue through a metal detection device and are then placed in cartons and sealed for shipment.

If the product of the present invention is a stuffed hollow celery stick product, the larger hollow celery stick variety sections are carried via conveyor from the sorting and grading conveyor to a filling/stuffing station. Here the sticks are filled using a stainless food grade injector and then conveyed to a packing station where they are packaged in appropriate packaging.

The sealer is again used for sealing, a metal detector is used to check for contamination and a boxing station places the containers into cartons for shipment.

If the stuffed hollow celery stick of the present invention is to be cooked the process breaks just after the filling station, depending on the specific product.

### Further Embodiments of the Invention

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as “transgenes”. Over the last fifteen to twenty years several methods for producing transgenic plants have been developed, and the present invention, in particular embodiments, also relates to transformed versions of the claimed line.

Plant transformation involves the construction of an expression vector that will function in plant cells. Such a vector comprises DNA comprising a gene under control of or operatively linked to a regulatory element (for example, a promoter). The expression vector may contain one or more such operably linked gene/regulatory element combinations. The vector(s) may be in the form of a plasmid, and can be used alone or in combination with other plasmids, to provide transformed celery plants, using transformation methods as described below to incorporate transgenes into the genetic material of the celery plant(s).

### Expression Vectors for Celery Transformation: Marker Genes

Expression vectors include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or a herbicide, or genes that encode an altered target which is insensitive to the inhibitor. A few positive selection methods are also known in the art.

One commonly used selectable marker gene for plant transformation is the neomycin phosphotransferase II (nptII) gene, isolated from transposon Tn5, which when placed under the control of plant regulatory signals which confers resistance to kanamycin (Fraley et al., Proc. Natl. Acad. Sci. U.S.A., 80:4803 (1983)). Another commonly used selectable marker gene is the hygromycin phosphotransferase gene which confers resistance to the antibiotic hygromycin (Vanden Elzen et al., Plant Mol. Biol., 5:299 (1985)).

Additional selectable marker genes of bacterial origin that confer resistance to antibiotics include gentamycin acetyl transferase, streptomycin phosphotransferase, aminoglycoside-3′-adenyl transferase, the bleomycin resistance determinant (Hayford et al., Plant Physiol. 86:1216 (1988), Jones et al., Mol. Gen. Genet, 210:86 (1987), Svab et al., Plant Mol. Biol. 14:197 (1990), Hille et al., Plant Mol. Biol. 7:171 (1986)). Other selectable marker genes confer resistance to herbicides such as glyphosate, glufosinate or bromoxynil (Comai et al., Nature 317:741-744 (1985), Gordon-Kamm et al., Plant Cell 2:603-618 (1990) and Stalker et al., Science 242:419-423 (1988)).

Selectable marker genes for plant transformation that are not of bacterial origin include, for example, mouse dihydrofolate reductase, plant 5-enolpyruvylshikimate-3-phosphate synthase and plant acetolactate synthase (Eichholtz et al., Somatic Cell Mol. Genet. 13:67 (1987), Shah et al., Science 233:478 (1986), Charest et al., Plant Cell Rep. 8:643 (1990)).

Another class of marker genes for plant transformation require screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include α-glucuronidase (GUS), α-galactosidase, luciferase and chloramphenicol, acetyltransferase (Jefferson, R. A., Plant Mol. Biol. Rep. 5:387 (1987), Teeri et al., EMBO J. 8:343 (1989), Koncz et al., Proc. Natl. Acad. Sci. U.S.A. 84:131 (1987), DeBlock et al., EMBO J. 3:1681 (1984)).

In vivo methods for visualizing GUS activity that do not require destruction of plant tissues are available (Molecular Probes publication 2908, IMAGENE GREEN, p. 1-4 (1993) and Naleway et al., J. Cell Biol. 115:151a (1991)). However, these in vivo methods for visualizing GUS activity have not proven useful for recovery of transformed cells because of low sensitivity, high fluorescent backgrounds and limitations associated with the use of luciferase genes as selectable markers.

More recently, a gene encoding Green Fluorescent Protein (GFP) has been utilized as a marker for gene expression in prokaryotic and eukaryotic cells (Chalfie et al., Science 263:802 (1994)). GFP and mutants of GFP may be used as screenable markers.

### Expression Vectors for Celery Transformation: Promoters

Genes included in expression vectors must be driven by a nucleotide sequence comprising a regulatory element, for example, a promoter. Several types of promoters are now well known in the transformation arts, as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters which initiate transcription only in certain tissue are referred to as “tissue-specific”. A “cell type” specific promoter primarily drives expression in certain cell types in one or more organs, for example, vascular cells in roots or leaves. An “inducible” promoter is a promoter which is under environmental control. Examples of environmental conditions that may effect transcription by inducible promoters include anaerobic conditions or the presence of light. Tissue-specific, tissue-preferred, cell type specific, and inducible promoters constitute the class of “non-constitutive” promoters. A “constitutive” promoter is a promoter which is active under most environmental conditions.

### A. Inducible Promoters

An inducible promoter is operably linked to a gene for expression in celery. Optionally, the inducible promoter is operably linked to a nucleotide sequence encoding a signal sequence which is operably linked to a gene for expression in celery. With an inducible promoter the rate of transcription increases in response to an inducing agent.

Any inducible promoter can be used in the instant invention. See Ward et al., Plant Mol. Biol. 22:361-366 (1993). Exemplary inducible promoters include, but are not limited to, that from the ACEI system which responds to copper (Meft et al., PNAS 90:4567-4571 (1993)); In2 gene from maize which responds to benzenesulfonamide herbicide safeners (Hershey et al., Mol. Gen. Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone. Schena et al., Proc. Natl. Acad. Sci. U.S.A. 88:0421 (1991).

### B. Constitutive Promoters

A constitutive promoter is operably linked to a gene for expression in celery or the constitutive promoter is operably linked to a nucleotide sequence encoding a signal sequence which is operably linked to a gene for expression in celery.

Many different constitutive promoters can be utilized in the instant invention. Exemplary constitutive promoters include, but are not limited to, the promoters from plant viruses such as the 35S promoter from CaMV (Odell et al., Nature 313:810-812 (1985) and the promoters from such genes as rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); PEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)). The ALS promoter, Xba1/NcoI fragment 5′ to the Brassica napus ALS3 structural gene (or a nucleotide sequence similarity to said Xba1/NcoI fragment), represents a particularly useful constitutive promoter. See PCT application WO 96/30530.

### C. Tissue-Specific or Tissue-Preferred Promoters

A tissue-specific promoter is operably linked to a gene for expression in celery. Optionally, the tissue-specific promoter is operably linked to a nucleotide sequence encoding a signal sequence which is operably linked to a gene for expression in celery. Plants transformed with a gene of interest operably linked to a tissue-specific promoter produce the protein product of the transgene exclusively, or preferentially, in a specific tissue.

Any tissue-specific or tissue-preferred promoter can be utilized in the instant invention. Exemplary tissue-specific or tissue-preferred promoters include, but are not limited to, a root-preferred promoter, such as that from the phaseolin gene (Murai et al., Science 23:476-482 (1983) and Sengupta-Gopalan et al., Proc. Natl. Acad. Sci. U.S.A. 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Signal Sequences for Targeting Proteins to Subcellular Compartments

Transport of protein produced by transgenes to a subcellular compartment such as the chloroplast, vacuole, peroxisome, glyoxysome, cell wall or mitochondrion or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized.

The presence of a signal sequence directs a polypeptide to either an intracellular organelle or subcellular compartment or for secretion to the apoplast. Many signal sequences are known in the art. See, for example Becker et al., Plant Mol. Biol. 20:49 (1992), Close, P. S., Master's Thesis, Iowa State University (1993), Knox, C., et al., “Structure and Organization of Two Divergent Alpha-Amylase Genes from Barley”, Plant Mol. Biol. 9:3-17 (1987), Lerner et al., Plant Physiol. 91:124-129 (1989), Fontes et al., Plant Cell 3:483-496 (1991), Matsuoka et al., Proc. Natl. Acad. Sci. 88:834 (1991), Gould et al., J. Cell. Biol. 108:1657 (1989), Creissen et al., Plant J. 2:129 (1991), Kalderon, et al., A short amino acid sequence able to specify nuclear location, Cell 39:499-509 (1984), Steifel, et al., Expression of a maize cell wall hydroxyproline-rich glycoprotein gene in early leaf and root vascular differentiation, Plant Cell 2:785-793 (1990).

### Foreign Protein Genes and Agronomic Genes

With transgenic plants according to the present invention, a foreign protein can be produced in commercial quantities. Thus, techniques for the selection and propagation of transformed plants, which are well understood in the art, yield a plurality of transgenic plants which are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981).

According to a preferred embodiment, the transgenic plant provided for commercial production of foreign protein is celery. In another preferred embodiment, the biomass of interest is seed. For the relatively small number of transgenic plants that show higher levels of expression, a genetic map can be generated, primarily via conventional RFLP, PCR and SSR analysis, which identifies the approximate chromosomal location of the integrated DNA molecule. For exemplary methodologies in this regard, see Glick and Thompson, Methods in Plant Molecular Biology and Biotechnology CRC Press, Boca Raton 269:284 (1993). Map information concerning chromosomal location is useful for proprietary protection of a subject transgenic plant. If unauthorized propagation is undertaken and crosses made with other germplasm, the map of the integration region can be compared to similar maps for suspect plants, to determine if the latter have a common parentage with the subject plant. Map comparisons would involve hybridizations, RFLP, PCR, SSR and sequencing, all of which are conventional techniques.

Likewise, by means of the present invention, agronomic genes can be expressed in transformed plants. More particularly, plants can be genetically engineered to express various phenotypes of agronomic interest. Exemplary genes implicated in this regard include, but are not limited to, those categorized below:

1. Genes that Confer Resistance to Pests or Disease and That Encode:

A. Plant disease resistance genes. Plant defenses are often activated by specific interaction between the product of a disease resistance gene (R) in the plant and the product of a corresponding avirulence (Avr) gene in the pathogen. A plant line can be transformed with a cloned resistance gene to engineer plants that are resistant to specific pathogen strains. See, for example Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al., Cell 78:1089 (1994) (Arabidopsis RSP2 gene for resistance to Pseudomonas syringae).

B. A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Nos. 40098, 67136, 31995 and 31998.

C. A lectin. See, for example, the disclosure by Van Damme et al., Plant Molec. Biol. 24:25 (1994), who disclose the nucleotide sequences of several Clivia miniata mannose-binding lectin genes.

D. A vitamin-binding protein such as avidin. See PCT application US 93/06487, the contents of which are hereby incorporated by reference. The application teaches the use of avidin and avidin homologues as larvicides against insect pests.

E. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor 1), Sumitani et al., Biosci. Biotech. Biochem. 57:1243 (1993) (nucleotide sequence of Streptomyces nitrosporeus α-amylase inhibitor).

F. An insect-specific hormone or pheromone such as an ecdysteroid and juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

G. An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor), and Pratt et al., Biochem. Biophys. Res. Comm. 163:1243 (1989) (an allostatin is identified in Diploptera puntata). See also U.S. Pat. No. 5,266,317 to Tomalski et al., who disclose genes encoding insect-specific, paralytic neurotoxins.

H. An insect-specific venom produced in nature by a snake, a wasp, etc. For example, see Pang et al., Gene 116:165 (1992), for disclosure of heterologous expression in plants of a gene coding for a scorpion insectotoxic peptide.

I. An enzyme responsible for a hyperaccumulation of a monoterpene, a sesquiterpene, a steroid, hydroxamic acid, a phenylpropanoid derivative or another non-protein molecule with insecticidal activity.

J. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hornworm chitinase, and Kawalleck et al., Plant Molec. Biol. 21:673 (1993), who provide the nucleotide sequence of the parsley ubi4-2 polyubiquitin gene.

K. A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol. 104:1467 (1994), who provide the nucleotide sequence of a maize calmodulin cDNA clone.

L. A hydrophobic moment peptide. See PCT application WO 95/16776 (disclosure of peptide derivatives of tachyplesin which inhibit fungal plant pathogens) and PCT application WO 95/18855 (teaches synthetic antimicrobial peptides that confer disease resistance), the respective contents of which are hereby incorporated by reference.

M. A membrane permease, a channel former or a channel blocker. For example, see the disclosure of Jaynes et al., Plant Sci 89:43 (1993), of heterologous expression of a cecropin-β, lytic peptide analog to render transgenic tobacco plants resistant to Pseudomonas solanacearum.

N. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. rev. Phytopathol. 28:451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

O. An insect-specific antibody or an immunotoxin derived therefrom. Thus, an antibody targeted to a critical metabolic function in the insect gut would inactivate an affected enzyme, killing the insect. Cf. Taylor et al., Abstract #497, Seventh Int'l Symposium on Molecular Plant-Microbe Interactions (Edinburgh, Scotland) (1994) (enzymatic inactivation in transgenic tobacco via production of single-chain antibody fragments).

P. A virus-specific antibody. See, for example, Tavladoraki et al., Nature 366:469 (1993), who show that transgenic plants expressing recombinant antibody genes are protected from virus attack.

Q. A developmental-arrestive protein produced in nature by a pathogen or a parasite. Thus, fungal endo-α-1,4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

R. A developmental-arrestive protein produced in nature by a plant. For example, Logemann et al., Bio/Technology 10:305 (1992), have shown that transgenic plants expressing the barley ribosome-inactivating gene have an increased resistance to fungal disease.

S. A lettuce mosaic potyvirus (LMV) coat protein gene introduced into Lactuca sativa in order to increase its resistance to LMV infection. See Dinant et al., Molecular Breeding. 1997, 3: 1, 75-86.

2. Genes that Confer Resistance to an Herbicide:

A. An herbicide that inhibits the growing point or meristem, such as an imidazolinone or a sulfonylurea. Exemplary genes in this category code for mutant ALS and AHAS enzyme as described, for example, by Lee et al., EMBO J. 7:1241 (1988), and Miki et al., Theor. Appl. Genet. 80:449 (1990), respectively.

B. Glyphosate (resistance conferred by mutant 5-enolpyruvlshikimate-3-phosphate synthase (EPSPS) and aroA genes, respectively) and other phosphono compounds such as glufosinate (phosphinothricin acetyl transferase (PAT) and Streptomyces hygroscopicus phosphinothricin-acetyl transferase PAT bar genes), and pyridinoxy or phenoxy proprionic acids and cyclohexones (ACCase inhibitor-encoding genes). See, for example, U.S. Pat. No. 4,940,835 to Shah, et al., which discloses the nucleotide sequence of a form of EPSPS which can confer glyphosate resistance. A DNA molecule encoding a mutant aroA gene can be obtained under ATCC accession number 39256, and the nucleotide sequence of the mutant gene is disclosed in U.S. Pat. No. 4,769,061 to Comai. See also Umaballava-Mobapathie in Transgenic Research. 1999, 8: 1, 33-44 that discloses Lactuca sativa resistant to glufosinate. European patent application No. 0 333 033 to Kumada et al., and U.S. Pat. No. 4,975,374 to Goodman et al., disclose nucleotide sequences of glutamine synthetase genes which confer resistance to herbicides such as L-phosphinothricin. The nucleotide sequence of a phosphinothricin-acetyl-transferase gene is provided in European application No. 0 242 246 to Leemans et al., DeGreef et al., Bio/Technology 7:61 (1989), describe the production of transgenic plants that express chimeric bar genes coding for phosphinothricin acetyl transferase activity. Exemplary of genes conferring resistance to phenoxy proprionic acids and cyclohexones, such as sethoxydim and haloxyfop are the Acc1-S1, Acc1-S2 and Acc1-S3 genes described by Marshall et al., Theor. Appl. Genet. 83:435 (1992).

C. An herbicide that inhibits photosynthesis, such as a triazine (psbA and gs+ genes) and a benzonitrile (nitrilase gene). Przibilla et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., Biochem. J. 285:173 (1992).

D. Acetohydroxy acid synthase, which has been found to make plants that express this enzyme resistant to multiple types of herbicides, has been introduced into a variety of plants. See Hattori et al., Mol. Gen. Genet. 246:419, 1995. Other genes that confer tolerance to herbicides include a gene encoding a chimeric protein of rat cytochrome P4507A1 and yeast NADPH-cytochrome P450 oxidoreductase (Shiota et al., Plant Physiol., 106:17, 1994), genes for glutathione reductase and superoxide dismutase (Aono et al., Plant Cell Physiol. 36:1687, 1995), and genes for various phosphotransferases (Datta et al., Plant Mol. Biol. 20:619, 1992).

E. Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306; 6,282,837; 5,767,373; and international publication WO 01/12825.

3. Genes that Confer or Contribute to a Value-Added Trait, Such as:

A. Increased iron content of the celery, for example by transforming a plant with a soybean ferritin gene as described in Goto et al., Acta Horticulturae. 2000, 521, 101-109.

B. Decreased nitrate content of leaves, for example by transforming a celery with a gene coding for a nitrate reductase. See for example Curtis et al., Plant Cell Report. 1999, 18: 11, 889-896.

C. Increased sweetness of the celery by transferring a gene coding for monellin, that elicits a flavor 100,000 times sweeter than sugar on a molar basis. See Penarrubia et al., Biotechnology. 1992, 10: 561-564.

D. Modified fatty acid metabolism, for example, by transforming a plant with an antisense gene of stearyl-ACP desaturase to increase stearic acid content of the plant. See Knultzon et al., Proc. Natl. Acad. Sci. USA 89:2625 (1992).

E. Modified carbohydrate composition effected, for example, by transforming plants with a gene coding for an enzyme that alters the branching pattern of starch. See Shiroza et al., J. Bacteriol. 170:810 (1988) (nucleotide sequence of Streptococcus mutants fructosyltransferase gene), Steinmetz et al., Mol. Gen. Genet. 20:220 (1985) (nucleotide sequence of Bacillus subtilis levansucrase gene), Pen et al., Bio/Technology 10:292 (1992) (production of transgenic plants that express Bacillus lichenifonnis α-amylase), Elliot et al., Plant Molec. Biol. 21:515 (1993) (nucleotide sequences of tomato invertase genes), Søgaard et al., J. Biol. Chem. 268:22480 (1993) (site-directed mutagenesis of barley α-amylase gene), and Fisher et al., Plant Physiol. 102:1045 (1993) (maize endosperm starch branching enzyme 11).

4. Genes that Control Male-Sterility

A. Introduction of a deacetylase gene under the control of a tapetum-specific promoter and with the application of the chemical N-Ac-PPT. See international publication WO 01/29237.

B. Introduction of various stamen-specific promoters. See international publications WO 92/13956 and WO 92/13957.

C. Introduction of the barnase and the barstar genes. See Paul et al., Plant Mol. Biol. 19:611-622, 1992).

### Methods for Celery Transformation

Numerous methods for plant transformation have been developed, including biological and physical, plant transformation protocols. See, for example, Miki et al., “Procedures for Introducing Foreign DNA into Plants” in Methods in Plant Molecular Biology and Biotechnology, Glick B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick B. R. and Thompson, J. E. Eds. (CRC Press, Inc., Boca Raton, 1993) pages 89-119.

### A. Agrobacterium-Mediated Transformation

One method for introducing an expression vector into plants is based on the natural transformation system of Agrobacterium. See, for example, Horsch et al., Science 227:1229 (1985), Curtis et al., Journal of Experimental Botany. 1994, 45: 279, 1441-1449, Torres et al., Plant cell Tissue and Organic Culture. 1993, 34: 3, 279-285, Dinant et al., Molecular Breeding. 1997, 3: 1, 75-86. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra, and Moloney et al., Plant Cell Reports 8:238 (1989). See also, U.S. Pat. No. 5,591,616 issued Jan. 7, 1997.

### B. Direct Gene Transfer

Several methods of plant transformation collectively referred to as direct gene transfer have been developed as an alternative to Agrobacterium-mediated transformation. A generally applicable method of plant transformation is microprojectile-mediated transformation wherein DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate plant cell walls and membranes. Russell, D. R., et al. PI. Cell. Rep. 12 (3, January), 165-169 (1993), Aragao, F. J. L., et al. Plant Mol. Biol. 20 (2, October), 357-359 (1992), Aragao, F. J. L., et al. PI. Cell. Rep. 12 (9, July), 483-490 (1993). Aragao, Theor. Appl. Genet. 93: 142-150 (1996), Kim, J.; Minamikawa, T. Plant Science 117: 131-138 (1996), Sanford et al., Part. Sci. Technol. 5:27 (1987), Sanford, J. C., Trends Biotech. 6:299 (1988), Klein et al., Bio/Technology 6:559-563 (1988), Sanford, J. C., Physiol Plant 7:206 (1990), Klein et al., Biotechnology 10:268 (1992).

Another method for physical delivery of DNA to plants is sonication of target cells. Zhang et al., Bio/Technology 9:996 (1991). Alternatively, liposome and spheroplast fusion have been used to introduce expression vectors into plants. Deshayes et al., EMBO J., 4:2731 (1985), Christou et al., Proc Natl. Acad. Sci. U.S.A. 84:3962 (1987). Direct uptake of DNA into protoplasts using CaCl₂ precipitation, polyvinyl alcohol or poly-L-ornithine have also been reported. Hain et al., Mol. Gen. Genet. 199:161 (1985) and Draper et al., Plant Cell Physiol. 23:451 (1982). Electroporation of protoplasts and whole cells and tissues have also been described. Saker, M.; Kuhne, T. Biologia Plantarum 40(4): 507-514 (1997/98), Donn et al., In Abstracts of VIIth International Congress on Plant Cell and Tissue Culture IAPTC, A2-38, p 53 (1990); D'Halluin et al., Plant Cell 4:1495-1505 (1992) and Spencer et al., Plant Mol. Biol. 24:51-61 (1994). See also Chupean et al., Biotechnology. 1989, 7: 5, 503-508.

Following transformation of celery target tissues, expression of the above-described selectable marker genes allows for preferential selection of transformed cells, tissues and/or plants, using regeneration and selection methods now well known in the art.

The foregoing methods for transformation would typically be used for producing a transgenic line. The transgenic line could then be crossed, with another (non-transformed or transformed) line, in order to produce a new transgenic celery line. Alternatively, a genetic trait which has been engineered into a particular celery cultivar using the foregoing transformation techniques could be moved into another line using traditional backcrossing techniques that are well known in the plant breeding arts. For example, a backcrossing approach could be used to move an engineered trait from a public, non-elite inbred line into an elite inbred line, or from an inbred line containing a foreign gene in its genome into an inbred line or lines which do not contain that gene. As used herein, “crossing” can refer to a simple X by Y cross, or the process of backcrossing, depending on the context.

### Single-Gene Conversions

When the term celery plant, cultivar or celery line are used in the context of the present invention, this also includes any single gene conversions of that line. The term “single gene converted plant” as used herein refers to those celery plants which are developed by a plant breeding technique called backcrossing wherein essentially all of the desired morphological and physiological characteristics of a cultivar are recovered in addition to the single gene transferred into the line via the backcrossing technique. Backcrossing methods can be used with the present invention to improve or introduce a characteristic into the line. The term “backcrossing” as used herein refers to the repeated crossing of a hybrid progeny back to one of the parental celery plants for that line, backcrossing 1, 2, 3, 4, 5, 6, 7, 8 or more times to the recurrent parent. The parental celery plant which contributes the gene for the desired characteristic is termed the nonrecurrent or donor parent. This terminology refers to the fact that the nonrecurrent parent is used one time in the backcross protocol and therefore does not recur. The parental celery plant to which the gene or genes from the nonrecurrent parent are transferred is known as the recurrent parent as it is used for several rounds in the backcrossing protocol (Poehiman & Sleper, 1994; Fehr, 1987). In a typical backcross protocol, the original cultivar of interest (recurrent parent) is crossed to a second line (nonrecurrent parent) that carries the single gene of interest to be transferred. The resulting progeny from this cross are then crossed again to the recurrent parent and the process is repeated until a celery plant is obtained wherein essentially all of the desired morphological and physiological characteristics of the recurrent parent are recovered in the converted plant, in addition to the single transferred gene from the nonrecurrent parent.

The selection of a suitable recurrent parent is an important step for a successful backcrossing procedure. The goal of a backcross protocol is to alter or substitute a single trait or characteristic in the original line. To accomplish this, a single gene of the recurrent cultivar is modified or substituted with the desired gene from the nonrecurrent parent, while retaining essentially all of the rest of the desired genetic, and therefore the desired physiological and morphological, constitution of the original line. The choice of the particular nonrecurrent parent will depend on the purpose of the backcross, one of the major purposes is to add some commercially desirable, agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many single gene traits have been identified that are not regularly selected for in the development of a new line but that can be improved by backcrossing techniques. Single gene traits may or may not be transgenic, examples of these traits include but are not limited to, male sterility, modified fatty acid metabolism, modified carbohydrate metabolism, herbicide resistance, resistance for bacterial, fungal, or viral disease, insect resistance, enhanced nutritional quality, industrial usage, yield stability and yield enhancement. These genes are generally inherited through the nucleus. Several of these single gene traits are described in U.S. Pat. Nos. 5,777,196, 5,948,957 and 5,969,212, the disclosures of which are specifically hereby incorporated by reference.

### Tissue Culture

Further reproduction of the variety can occur by tissue culture and regeneration. Tissue culture of various tissues of celery and regeneration of plants therefrom is well known and widely published. For example, reference may be had to Teng et al., HortScience. 1992, 27: 9, 1030-1032 Teng et al., HortScience. 1993, 28: 6, 669-1671, Zhang et al., Journal of Genetics and Breeding. 1992, 46: 3, 287-290, Webb et al., Plant Cell Tissue and Organ Culture. 1994, 38: 1, 77-79, Curtis et al., Journal of Experimental Botany. 1994, 45: 279, 1441-1449, Nagata et al., Journal for the American Society for Horticultural Science. 2000, 125: 6, 669-672, and Ibrahim et al., Plant Cell, Tissue and Organ Culture. (1992), 28(2): 139-145. It is clear from the literature that the state of the art is such that these methods of obtaining plants are routinely used and have a very high rate of success. Thus, another aspect of this invention is to provide cells which upon growth and differentiation produce celery plants having the physiological and morphological characteristics of an edible hollow celery stick.

As used herein, the term “tissue culture” indicates a composition comprising isolated cells of the same or a different type or a collection of such cells organized into parts of a plant. Exemplary types of tissue cultures are protoplasts, calli, meristematic cells, and plant cells that can generate tissue culture that are intact in plants or parts of plants, such as leaves, pollen, embryos, roots, root tips, anthers, pistils, flowers, seeds, petioles, suckers and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference.

### Additional Breeding Methods

This invention also is directed to methods for producing a celery plant by crossing a first parent celery plant with a second parent celery plant wherein the first or second parent celery plant is a celery plant of an edible, hollow celery stick. Further, both first and second parent celery plants can come from an edible, hollow celery stick. Thus, any such methods using an edible, hollow celery stick are part of this invention: selfing, backcrosses, hybrid production, crosses to populations, and the like. All plants produced using an edible, hollow celery stick as at least one parent are within the scope of this invention, including those developed from cultivars derived from an edible hollow celery stick. Advantageously, this celery cultivar could be used in crosses with other, different, celery plants to produce the first generation (F₁) celery hybrid seeds and plants with superior characteristics. The cultivar of the invention can also be used for transformation where exogenous genes are introduced and expressed by the cultivar of the invention. Genetic variants created either through traditional breeding methods using an edible, hollow celery stick or through transformation of an edible hollow celery stick by any of a number of protocols known to those of skill in the art are intended to be within the scope of this invention.

The following describes breeding methods that may be used with an edible, hollow celery stick in the development of further celery plants. One such embodiment is a method for developing an edible, hollow celery stick progeny celery plants in a celery plant breeding program comprising: obtaining the celery plant, or a part thereof, of an edible, hollow celery stick utilizing said plant or plant part as a source of breeding material, and selecting an edible, hollow celery stick progeny plant with molecular markers in common with an edible, hollow celery stick. Breeding steps that may be used in the celery plant breeding program include pedigree breeding, back crossing, mutation breeding, and recurrent selection. In conjunction with these steps, techniques such as RFLP-enhanced selection, genetic marker enhanced selection (for example SSR markers) and the making of double haploids may be utilized.

Another method involves producing a population of an edible, hollow celery stick progeny celery plants, comprising crossing an edible, hollow celery stick with another celery plant, thereby producing a population of celery plants, which, on Mean, derive 50% of their alleles from an edible, hollow celery stick. A plant of this population may be selected and repeatedly selfed or sibbed with a celery cultivar resulting from these successive filial generations. One embodiment of this invention is the celery cultivar produced by this method and that has obtained at least 50% of its alleles from an edible, hollow celery stick.

One of ordinary skill in the art of plant breeding would know how to evaluate the traits of two plant varieties to determine if there is no significant difference between the two traits expressed by those varieties. For example, see Fehr and Walt, Principles of Cultivar Development, p 261-286 (1987). Thus the invention includes an edible, hollow stick progeny celery plants comprising a combination of at least two edible, hollow celery stick traits selected from the group consisting of the edible, hollow celery stick combination of traits listed in the Summary of the Invention, so that said progeny celery plant is not significantly different for said traits than edible, hollow celery stick as determined at the 5% significance level when grown in the same environmental conditions. Using techniques described herein, molecular markers may be used to identify said progeny plant as edible, hollow celery stick progeny plant. Mean trait values may be used to determine whether trait differences are significant, and preferably the traits are measured on plants grown under the same environmental conditions. Once such a variety is developed its value is substantial since it is important to advance the germplasm base as a whole in order to maintain or improve traits such as yield, disease resistance, pest resistance, and plant performance in extreme environmental conditions.

Progeny of edible, hollow celery stick may also be characterized through their filial relationship with edible, hollow celery sticks, as for example, being within a certain number of breeding crosses of edible, hollow celery stick. A breeding cross is a cross made to introduce new genetics into the progeny, and is distinguished from a cross, such as a self or a sib cross, made to select among existing genetic alleles. The lower the number of breeding crosses in the pedigree, the closer the relationship between the edible, hollow celery stick and its progeny. For example, progeny produced by the methods described herein may be within 1, 2, 3, 4 or 5 breeding crosses of edible, hollow celery stick.

As used herein, the term “plant” includes plant cells, plant protoplasts, plant cell tissue cultures from which celery plants can be regenerated, plant calli, plant clumps and plant cells that are intact in plants or parts of plants, such as leaves, pollen, embryos, roots, root tips, anthers, pistils, flowers, seeds, petioles, suckers and the like.

### Deposit Information

A deposit of the A. Duda & Sons, Inc. proprietary inbred celery cultivar ADS-9 disclosed above and recited in the appended claims has been made with the American Type Culture Collection (ATCC), 10801 University Boulevard, Manassas, Va. 20110. The date of deposit was Jun. 17, 2004. The deposit of 2,500 seeds was taken from the same deposit maintained by A. Duda & Sons, Inc. since prior to the filing date of this application. All restrictions upon the deposit have been removed, and the deposit is intended to meet all of the requirements of 37 C.F.R. 1.801-1.809. The ATCC accession number is PTA-6083. The deposit will be maintained in the depository for a period of 30 years, or 5 years after the last request, or for the effective life of the patent, whichever is longer, and will be replaced as necessary during that period.

A deposit of the celery cultivar seed of ADS-15 and ADS-19 of this invention is maintained by A. Duda & Sons, Inc., 1260 Growers Street, Salinas, Calif. 93902, U.S.A. Access to this deposit will be available during the pendency of this application to persons determined by the Commissioner of Patents and Trademarks to be entitled thereto under 37 CFR §1.14 and 35 USC §122. Upon allowance of any claims in this application, all restrictions on the availability to the public of the variety will be irrevocably removed by affording access to a deposit of at least 2,500 seeds of the same variety with the American Type Culture Collection (ATCC), 10801 University Boulevard, Manassas, Va. 20110 or National Collections of Industrial, Food and Marine Bacteria (NCIMB), 23 St Machar Drive, Aberdeen, Scotland, AB24 3RY, United Kingdom.

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

What is claimed is:

1. An Apium graveolens L. var dulce celery plant with a hollow petiole.

2. The hollow petiole of claim 1, wherein said hollow petiole is cut into at least one celery stick between 2.0 and 36.0 centimeters in length.

3. The celery stick of claim 2, wherein the celery stick is stuffed with consumable products into said hollow petiole.

4. A method for producing a hollow celery stick from an Apium graveolens L. var dulce celery plant, comprising the steps of
a. cutting said celery plant to remove leaves;
b. cutting said celery plant to remove butt of celery;
c. removing heart of said celery plant;
d. cutting said celery plant into a celery stick between 2.0 and 36.0 centimeters; and
e. sanitizing said celery stick to produce a celery stick;

5. A celery stick produced by the method of claim 4, wherein said cutting said celery is performed by a means selected from the group consisting of knives, razor sharp blades, saws, water jets, lasers and sound waves.

6. A celery stick produced by the method of claim 4, wherein sanitizing said celery is performed by a sanitization treatment selected from the group consisting of ascorbic acid, peroxyacetic acid, sodium hypochlorite, chlorine, bromine, sodium hypobromine, chlorine dioxide, ozone based systems, hydrogen peroxide products, trisodium phosphate, quaternary ammonium products, ultraviolet light systems, irradiation, steam, ultra heat treatments, and high pressure pasteurization.

7. A celery stick produced by the method of claim 4, wherein said celery stick is packaged in a package selected from the group consisting of flexible film, rigid plastic, solid fiber, poly sleeves, plastic sleeves, poly bags, plastic bags, natural decomposable bags, natural decomposable sleeves, rigid containers, clam shells, packages with built-in vents and packages with specialized pores.

8. A method of injecting consumable material into the Apium graveolens L. var dulce celery stick of claim 4 to produce a stuffed celery stick, comprising the steps of:
a. connecting a reservoir containing pressurized consumable material to an injection device;
b. positioning said device into a said hollow celery stick; and
c. injecting into said celery stick consumable material from said injection device to deliver a quantity of said consumable material.

9. A celery stick produced by the method of claim 8.

10. The celery stick produced by the method claim 8, wherein said consumable material is selected from the group consisting of dairy based products, synthetic food types, nut based fillings, soy based products, chocolate, fruits and vegetable products, candy products, ethnic flavorings such as products with Mexican, Japanese, Chinese or Indian flavors or spices, fillings with preservatives, amendments to modify textures such as starches or to control moisture levels, products with nutritional fortification, minerals, calcium, potassium and vitamins.

11. The celery stick of claim 9, wherein said celery stick is battered, coated and frozen.

12. The celery stick of claim 10, wherein said celery stick is battered, coated and frozen.

13. The celery stick of claim 9, wherein said celery stick is grilled, baked or fried.

14. The celery stick of claim 12, wherein said celery stick is grilled, baked or fried.

15. The Apium graveolens L. var dulce celery petiole of claim 1, wherein said petiole has a petiole width between 8.0 mm and 22.0 mm.

16. The Apium graveolens L. var dulce celery petiole of claim 1, wherein said petiole has a petiole depth between 6.5 mm and 18.5 mm.

17. The Apium graveolens L. var dulce celery petiole of claim 1, wherein said petiole has a hollow petiole with an ability to withstand a vacuum pressure between 12.0 in/hg and 29.0 in/g.

18. The Apium graveolens L. var dulce celery petiole of claim 1, wherein said petiole has a wall thickness at the inside petiole cup tissue between 0.67 mm and 2.89 mm.

19. The Apium graveolens L. var dulce celery petiole of claim 1, wherein said petiole has a wall thickness at the sidewall of the petiole between 1.50 mm and 5.00 mm.

20. The Apium graveolens L. var dulce celery petiole of claim 1, wherein said petiole has an inside petiole cup tissue with the ability to withstand pressure between 300 grams of pressure and 1300 grams of pressure.
